(19) United States
(12) Patent Application Publication
(10) Pub. No.: US 2020/0197499 A1
(43) Pub. Date: Jun. 25, 2020

(54) Yeast-Based Immunotherapy for Tumour Prevention

(71) Applicant: INOVACTIS, Evry Cedex (FR)

(72) Inventors: Clément DE OBALDIA, Sceaux (FR); Pierre-Yves NOGUE, Vaucresson (FR)

(21) Appl. No.: 16/500,179

(22) PCT Filed: Apr. 4, 2018
(86) PCT No.: PCT/FR2018/050837

§ 371 Date: Oct. 2, 2019

(30) Foreign Application Priority Data
Apr. 4, 2017 (FR) 1752914

(51) Int. Cl. A61K 39/00; C12N 1/06; C12N 1/18; C07K 16/28

(52) U.S. Cl. CPC A61K 39/001186; C12N 1/063; A61K 39/0002; A61K 2039/876; C07K 16/2851; A61K 39/001156; A61K 2039/523; C12N 1/18

(57) ABSTRACT

The present invention relates to a process for preparing an immunotherapeutic yeast, said immunotherapeutic yeast expressing one or more tumor antigen(s) at its wall, and also to immunotherapeutic yeasts capable of being obtained by carrying out the process of the invention.

The present invention relates to a process for preparing an immunotherapeutic yeast, said immunotherapeutic yeast expressing one or more tumor antigen(s) at its wall, and also to immunotherapeutic yeasts capable of being obtained by carrying out the process of the invention.

### PRIOR ART

The treatment of cancer is a field that brings together different therapeutic approaches, including immunotherapies. Among immunotherapies, a distinction is made between passive immunotherapies and active immunotherapies, the latter possibly being targeted or untargeted. One of the advantages of active immunotherapies lies in the fact that they can cross the blood-brain barrier, unlike chemotherapy-type therapies.

Indeed, it has been demonstrated that lymphocytes were capable of migrating across the blood-brain barrier (Larochelle C., Alvarez J I, Prat A. How do immune cells overcome the blood-brain barrier in multiple sclerosis. FEBS Lett. 2011 Dec. 1; 585 (23):3770-80).

Aside from this advantage of crossing the blood-brain barrier, immunotherapies generally have fewer adverse effects than traditional anti-cancer therapies, which is particularly borne out for targeted immunotherapies. This is why immunotherapies are particularly suitable in the treatment of high-grade stage III or IV metastatic cancers in already highly weakened patients.

Untargeted active immunotherapies, such as anti-PD-1 antibodies and/or anti-CTLA4 antibodies, significantly improve the median survival of patients. However, these treatments benefit a small percentage of patients and are accompanied by significant immunological side effects such as autoimmune diseases. To overcome the risks of autoimmune diseases and improve their effectiveness, targeted active immunotherapies directed against a defined tumor antigen have been developed. Dendritic cells are major players in these new immunotherapies because they coordinate both an innate immune response and an adaptive response against cancer cells. One of the objectives of targeted active immunotherapies is therefore to stimulate these dendritic cells to generate tumor-reactive T lymphocytes, killer T lymphocytes or cytotoxic T lymphocytes. The action of these T lymphocytes is to induce both a regression of the size of the tumor, which can go as far as the disappearance of the tumor, and to induce immunological memory, limiting relapses.

The yeast Saccharomyces cerevisiae has been successfully used as a vector for targeted active immunotherapies because it constitutes an excellent adjunct for the induction of dendritic cells, which in turn activate cytotoxic T lymphocytes to destroy the cancer cells. To enable the activation of dendritic cells, the yeast must produce the tumor antigen to be targeted. U.S. Pat. No. 5,830,463 describes the use of a non-pathogenic yeast, genetically modified to express at least one compound capable of modulating the immune response and demonstrates that this genetically modified yeast is effective in stimulating both the cellular response and the humoral response when the yeast is administered to a mammal. U.S. Pat. No. 8,734,778 describes yeasts capable of expressing various tumor antigens in their cytosol, said whole yeasts being killed by heat before injection. Patent application US2008/0171059 describes the use of yeasts expressing a tumor antigen at their surface rather than in the cytosol.

Nevertheless, there is a need for new anti-cancer therapies that are effective and/or that have lesser undesirable side effects, especially compared to existing immunotherapeutic yeasts.

The inventors have observed, unexpectedly, that a yeast genetically modified to express one or more tumor antigen(s) at its wall and that has been permeabilized makes it possible to effectively stimulate cytotoxic T lymphocytes against the tumor.

### SUMMARY OF THE INVENTION

A subject of the present invention is a process for preparing an immunotherapeutic yeast, said process comprising the steps of:
a) obtaining a genetically modified yeast which expresses, at its wall, one or more tumor antigen(s) and optionally a polypeptide or a protein for targeting dendritic cells;
b) permeabilizing the genetically modified yeast in order to obtain an immunotherapeutic yeast, optionally inactivating the yeast before or after the permeabilization step.

Another subject of the invention consists of an immunotherapeutic yeast able to be obtained by carrying out the preparation process according to the invention.

Another subject of the invention relates to a genetically modified and permeabilized immunotherapeutic yeast which expresses, at its wall:
(i) one or more tumor antigen(s), preferably chosen from Melan-A, Tyrosinase, gp100, MAGE-B1, MAGEA1, MAGEA10, MAGEA11, MAGEA12, MAGEA2, MAGEA2B, MAGEA3, MAGEA4, MAGEA6, MAGEA8, MAGEA9, MAGEB1; MAGEB10, MAGEB16, MAGEB18, MAGEB2, MAGEB3, MAGEB4, MAGEB5, MAGEB6, MAGEB6B, New York Esophageal 1 antigen (NY-ESO-1), MAGEC1, MAGEC2, L-antigen (LAGE), TRP-1, TRP-2, P53, KRAS, CEA, WT1, MUC1, SART3, SURVIVIN 2B, RNF43/TOMM34, TGFBRII, HER2/neu, BRAF, PI3K, APC, BAX, beta-2 microglobulin, telomerase or NRAS; and
(ii) optionally a polypeptide or protein for targeting dendritic cells.

Another subject of the invention consists of an immunotherapeutic composition comprising an immunotherapeutic yeast according to the invention and a pharmaceutically acceptable carrier.

Another subject of the invention relates to a yeast according to the invention or a composition according to the invention for use thereof as medicament, especially for use thereof in the treatment or prevention of cancer.

### DETAILED DESCRIPTION

### Definitions

“Peptide” is intended to mean an amino acid sequence which generally comprises from 2 to 9 amino acids. “Polypeptide” is intended to mean an amino acid sequence which generally comprises from 10 to 100 amino acids. “Protein” is intended to mean an amino acid sequence which generally comprises more than 100 amino acids.

“Yeast” is intended to mean any single-celled fungus or any eukaryotic microorganism composed of a cell wall, of a cytoplasmic membrane, of a nucleus and of mitochondria, capable of asexual reproduction by budding or division. Among the well-known yeasts are Ascomycetes (Saccharomyces, Kluyveromyces, Pichia, Hansenula), Basidiomycetes (Sporobolomyces) and Deuteromycetes. The term “yeast(s)” is understood equally in the singular and in the plural.

“Genetically modified yeast” is intended to mean a yeast, the genetic heritage of which is artificially modified by introducing one or more nucleic sequences (or transgene(s)), transiently or definitively, so as to produce one or more peptides, polypeptides and/or proteins in said yeast. The transgenes may be derived from nucleic sequences of the same species as the genetically modified yeast (e.g. the anchoring polypeptide), of another species of yeast than the genetically modified yeast (e.g. the anchoring polypeptide) or of another prokaryotic or eukaryotic species (e.g. the tumor antigen). In the context of the invention, the transgene may, for example, encode one or more fusion protein(s) of formula (Ia):

[protein or polypeptide for targeting dendritic cells]ₙ-[tumor antigen]ₓ-[peptide linker]ₘ-[polypeptide or protein for anchoring to the wall of the genetically modified yeast] (Ia);

n is equal to 0 or 1, m is equal to 0 or 1, and x is an integer ranging from 1 to 300, preferably ranging from 1 to 50, preferably ranging from 1 to 9, preferably ranging from 1 to 5.

Thus, a “genetically modified yeast which expresses, at its wall, one or more tumor antigen(s)” is a yeast, the genetic heritage of which is artificially modified by the introduction of one or more nucleic sequence(s) encoding one or more tumor antigen(s) at the wall of said yeast, for example encoding a fusion protein of formula (Ia). In the present description, the terms “genetically modified yeast” and “modified yeast” are interchangeable.

“Tumor antigen(s)” is intended to mean all or part of a protein originating from a tumor protein and able to trigger a humoral and/or cellular immune response against a tumor. Advantageously, the tumor antigen(s) is/are chosen from Melan-A, Tyrosinase, gp100, MAGE-B1, MAGEA1, MAGEA10, MAGEA11, MAGEA12, MAGEA2, MAGEA2B, MAGEA3, MAGEA4, MAGEA6, MAGEA8, MAGEA9, MAGEB1; MAGEB10, MAGEB16, MAGEB18, MAGEB2, MAGEB3, MAGEB4, MAGEB5, MAGEB6, MAGEB6B, New York Esophageal 1 antigen (NY-ESO-1), MAGEC1, MAGEC2, L-antigen (LAGE), TRP-1, TRP-2, P53, KRAS, CEA, WT1, MUC1, SART3, SURVIVIN 2B, RNF43/TOMM34, TGFBRII, HER2/neu, BRAF, PI3K, APC, BAX, beta-2 microglobulin, telomerase or NRAS.

Ovalbumin, which is not a tumor antigen for the purposes of the invention, is used as model antigen in a great number of experiments, since the epitopes of the protein ovalbumin bind to MHC I and MHC II in mice. The epitopes of ovalbumin are among the best characterized for their very specific immunogenicity, that is to say their ability to trigger a targeted immune response against these epitopes. To characterize a specific in vivo immune response, there are OT-1 transgenic mice, the T lymphocytes of which are OVA-specific and specifically recognize OVA residues 257-264. The specific immune response can also be studied in vitro with the B3Z hybridoma cell line of T lymphocytes specific for the OVA 257-264 epitope.

“Expressed at the wall” is intended to mean the fact of being presented at the wall of the yeast. In the context of the invention, an antigen expressed at the wall is an antigen anchored to the wall of the yeast, for example via an anchoring protein or polypeptide.

“Permeabilized yeast” is intended to mean a yeast which has undergone a chemical, enzymatic or mechanical treatment aiming to permeabilize (or perforate) its wall and/or its cytoplasmic membrane. The means for permeabilizing the wall and/or the cytoplasmic membrane of a yeast are well known to those skilled in the art and can be used without particular difficulty to obtain the permeabilized yeasts. The permeabilization of a whole yeast enables access to the proteins contained in its cytosol (Optimization of permeabilization process of yeast cells for catalase activity using response surface methodology, Trawczynska et al, 2015) and/or in its periplasm. Treatments that make it possible to permeabilize the cytoplasmic membrane modify the cytoplasmic membrane of the yeast by forming pores that enable the free diffusion of small molecules such as enzyme substrates or products, but that maintain most of the proteins in the cytosol (Parmjit S. Panesar, Reeba Panesar, Ram S. Singh and Manav B. Bera, 2007. Permeabilization of Yeast Cells with Organic Solvents for β-galactosidase Activity. Research Journal of Microbiology, 2: 34-41). The wall of the yeast is naturally permeable to proteins up to approximately 70 kDa. Thus, the term “permeabilize” according to the invention is also intended to mean an increase in the permeability of the wall of the yeast.

For example, it is possible to determine whether a yeast is permeabilized or not by measuring the absorbance at 405 nm of a yeast incubated with para-nitrophenyl phosphate (pNPP) for 30 min. Under these conditions, a yeast is permeabilized when it has an absorbance at 405 nm of greater than or equal to 0.1, for example greater than or equal to 0.2. pNPP is the substrate of an enzyme, alkaline phosphatase, which is naturally present in the cytosol of the yeast. pNPP does not penetrate into the cytosol of a non-permeabilized yeast. On the other hand, when the yeast is permeabilized, the pNPP can enter the yeast and be hydrolyzed by the alkaline phosphatase present in the cytosol. The hydrolyzed pNPP then assumes a yellow color which can be read by absorbance at 405 nm (A rapid method for determination of acid phosphatase activity of whole yeast cells, Galabova et al, June 2008, Letters in Applied Microbiology 16 (3):161 - 163). Example 9 presents the detailed protocol of a test that makes it possible to determine whether a yeast is permeabilized or not.

“Immunotherapeutic yeast” is intended to mean a yeast capable of inducing a humoral or cellular immune response in humans or animals, thus making it possible to treat or prevent cancer.

### Process for Preparing the Yeasts

A subject of the present invention is a process for preparing an immunotherapeutic yeast, said process comprising the steps of:
a) obtaining a genetically modified yeast which expresses, at its wall, one or more tumor antigen(s) and optionally a polypeptide or a protein for targeting dendritic cells;
b) permeabilizing the genetically modified yeast in order to obtain an immunotherapeutic yeast, optionally inactivating the yeast before or after the permeabilization step.

When the process comprises an inactivation step, the steps of inactivation and of permeabilization can be carried out in any order.

Thus, according to a particular embodiment, the process according to the invention comprises the steps of:
a) obtaining a genetically modified yeast which expresses, at its wall, one or more tumor antigen(s) and optionally a polypeptide or a protein for targeting dendritic cells;
b) inactivating the genetically modified yeast; and
c) permeabilizing the inactivated genetically modified yeast in order to obtain an immunotherapeutic yeast.

According to another particular embodiment, the process according to the invention comprises the steps of:
a) obtaining a genetically modified yeast which expresses, at its wall, one or more tumor antigen(s) and optionally a polypeptide or a protein for targeting dendritic cells;
b) permeabilizing the genetically modified yeast; and
c) inactivating the permeabilized genetically modified yeast in order to obtain an immunotherapeutic yeast.

Advantageously, the tumor antigen(s) expressed on the wall of the yeast are oriented toward the external environment of the yeast and not toward the periplasm of the yeast. Orientation toward the outside of the yeast enables, after phagocytosis of the yeasts, quicker degradation of the tumor antigens by the proteases of the dendritic cells, which thus facilitates the cross-presentation of the tumor antigens to the killer lymphocytes (Howland, Wittrup, Antigen Release Kinetics in the Phagosome Are Critical to Cross-Presentation Efficiency, J Immunol. 2008 Feb. 1; 180 (3): 1576).

The inactivation step aims to inactivate the yeasts. “Inactivate a yeast” is intended to mean a treatment which consists in stopping growth by cell division of the yeast. The inactivation of a yeast can be carried out by any means known to those skilled in the art for inactivating microorganisms. Advantageously, the inactivation also makes it possible to fix the yeast. Advantageously, the yeast is inactivated with paraformaldehyde (PFA) at an amount of 0.5% in PBS (v/v).

The permeabilization step is carried out by means of a chemical, enzymatic or mechanical treatment, resulting in the permeabilization of the wall and/or the cytoplasmic membrane. In a particular embodiment, the genetically modified yeast is permeabilized with a solvent, for example a polar solvent. The solvent may be chosen from ethanol, sorbitol, 2-mercaptoethanol, benzene, n-butanol, n-propanol, triton X-100, isopropanol, methanol, toluene, acetone or a mixture of lithium acetate and sodium hydroxide. In another particular embodiment, the genetically modified yeast is permeabilized with an enzyme, for example β-1,3-glucanase (βGlu). In another particular embodiment, the genetically modified yeast is mechanically permeabilized by subjecting the yeast to freeze/thaw cycles (Hong-wei Zhao et al, 2011, Biotechnology & Biotechnological Equipment). In a preferred embodiment, the genetically modified yeast is permeabilized with ethanol or isopropanol, for example with a mixture of 50% ethanol and 50% water (volume to volume; v/v), for example for approximately 15 minutes, approximately 20 minutes or approximately 25 minutes.

In a particular embodiment of the process according to the invention, step a) is: obtaining a genetically modified yeast which expresses, at its wall, one or more fusion proteins of formula (Ia):

[protein or polypeptide for targeting dendritic cells]ₙ-[tumor antigen]ₓ-[peptide linker]ₘ-[polypeptide or protein for anchoring to the wall of the genetically modified yeast] (Ia);

n is equal to 0 or 1, m is equal to 0 or 1, and x is an integer ranging from 1 to 300, preferably ranging from 1 to 50, preferably ranging from 1 to 9, preferably ranging from 1 to 5, for example from 1 to 4, from 1 to 3, from 1 to 2, for example equal to 1, equal to 2, equal to 3, equal to 4, equal to 5.

In the context of the present description, when x is an integer greater than 1, that is to say when x is an integer ranging from 2 to 300, the tumor antigens may be identical or different. For example, the tumor antigens may all be different.

In the context of the present description, when x is an integer greater than 1, that is to say when x is an integer ranging from 2 to 300, the tumor antigens may be separated or not by a linker peptide.

In the context of the present description, when x is an integer greater than 1, that is to say when x is an integer ranging from 2 to 300, the tumor antigens may be identical or different. For example, when x=2, the tumor antigens may be two MAGEA1 antigens or one MAGEA1 antigen and one MAGEA2 antigen. In this embodiment, the tumor antigens may be placed one after the other or separated by a peptide sequence that connects said tumor antigens to one another.

Fusion proteins are widely described in the prior art. A “fusion protein” is a protein obtained by the combination of different peptides, polypeptides and/or proteins. Fusion proteins may also be referred to as chimeric proteins.

In a particular embodiment, step a) comprises the steps of:
a1) introducing, into a yeast, one or more vector(s) each comprising a nucleic sequence of formula (IIa) or (IIb):

[nucleic sequence encoding a polypeptide for addressing the wall of the genetically modified yeast]-[nucleic sequence encoding a polypeptide or a protein for targeting dendritic cells]ₙ-[nucleic sequence encoding a tumor antigen]ₓ-[nucleic sequence encoding a peptide linker]ₘ-[nucleic sequence encoding a polypeptide or a protein for anchoring to the wall of the genetically modified yeast] (IIa),

n is equal to 0 or 1, m is equal to 0 or 1, and x is an integer ranging from 1 to 300, preferably ranging from 1 to 50, preferably ranging from 1 to 9, preferably ranging from 1 to 5, for example from 1 to 4, from 1 to 3, from 1 to 2, for example equal to 1, equal to 2, equal to 3, equal to 4, equal to 5;

[nucleic sequence encoding a polypeptide or a protein for targeting dendritic cells]ₙ-[nucleic sequence encoding a tumor antigen]ₓ-[nucleic sequence encoding a peptide linker]ₘ-[nucleic sequence encoding a polypeptide or a protein for anchoring to the wall of the genetically modified yeast]-[nucleic sequence encoding a polypeptide for addressing the wall of the genetically modified yeast] (IIb),

n is equal to 0 or 1, m is equal to 0 or 1, and x is an integer ranging from 1 to 300, preferably ranging from 1 to 50, preferably ranging from 1 to 9, preferably ranging from 1 to 5, for example from 1 to 4, from 1 to 3, from 1 to 2, for example equal to 1, equal to 2, equal to 3, equal to 4, equal to 5;

in order to obtain a genetically modified yeast capable of expressing, at its wall, one or more fusion protein(s) of formula (Ia):

[protein or polypeptide for targeting dendritic cells]ₙ-[tumor antigen]ₓ-[peptide linker]ₘ-[polypeptide or protein for anchoring to the wall of the genetically modified yeast] (Ia),

n is equal to 0 or 1, m is equal to 0 or 1, and x is an integer ranging from 1 to 300, preferably ranging from 1 to 50, preferably ranging from 1 to 9, preferably ranging from 1 to 5, for example from 1 to 4, from 1 to 3, from 1 to 2, for example equal to 1, equal to 2, equal to 3, equal to 4, equal to 5; and
a2) culturing the genetically modified yeast under conditions suitable for the expression of the fusion protein(s) at the wall of the genetically modified yeast.

For the purposes of the present invention, the term “vector” should be taken in its broadest sense. In particular, a “vector” designates a carrier used to introduce a nucleotide sequence into a yeast, for expression thereof, for replication thereof and/or for integration thereof into the genome of said yeast. The different types of vectors are widely known to those skilled in the art and all types of vectors can be used in the context of the present invention. The vector(s) are also referred to as plasmid(s). The plasmid(s) may be linearized to allow homologous recombination in yeast, as explained in the examples.

The different nucleic sequences can be assembled in a vector by the Golden Gate technique. (Engel et al., 2008, PLos One and EP2395087). This technique enables simultaneous and directional digestion and assembly of multiple nucleic sequences in a single reaction, using Type IIs restriction enzymes, endonucleases that recognize particular sites then cleave nucleic sequences outside their recognition site. The introduction of the nucleic sequences (i.e. of the vector(s)) into the genome of the yeast is carried out in a replicative mode or in an integrative mode. In an integrative mode, the nucleic sequences are inserted into the genome of the yeast in a stable manner. In a replicative mode, the nucleic sequences are inserted into the yeast transiently using a replicative plasmid.

The “polypeptide for addressing the wall of the genetically modified yeast” or “addressing polypeptide” makes it possible to address the fusion protein (Ia) to the wall of the genetically modified yeast. This addressing to the wall then enables the fusion protein to anchor itself to the wall of the genetically modified yeast. Advantageously, the nucleic sequence encoding the addressing polypeptide is located at the 3′ end of the nucleic sequence (IIa) or (IIb) encoding the fusion protein (Ia). Thus, the addressing polypeptide is advantageously at the N-terminus of the fusion protein (Ia). In a particular embodiment, the addressing polypeptide is naturally present in the anchoring protein or polypeptide (for example in the Aga2p sequence). In another particular embodiment, the addressing polypeptide is artificially added to the nucleic sequence encoding the fusion protein (Ia) when the anchoring protein or polypeptide does not naturally comprise an addressing polypeptide (for example in the Sed1p sequence). In a particular embodiment, when the polypeptide or anchoring protein does not naturally comprise an addressing polypeptide, the addressing polypeptide is the “prepro alpha factor leader peptide” derived from the yeast pheromone (Mf(alpha)1p). The nucleic sequence encoding Mf(alpha)1p is SEQ ID No. 12.

Step a2) may be carried out in any culture medium ensuring the viability and reproducibility of the yeast. Yeast culture media are well known to those skilled in the art. Mention may be made for example of a glucose medium or a galactose medium.

In a particular embodiment, the vector(s) comprise a nucleic sequence encoding a peptide linker which forms the link between the anchoring polypeptide or protein and the tumor antigen(s). Thus, in this embodiment, the nucleic sequence (IIa) or (IIb) comprises a nucleic sequence encoding the peptide linker (i.e. m=1 for the nucleic sequence encoding the peptide linker).

“Peptide linker” or “peptide linker arm” is intended to mean an amino acid sequence that connects a polypeptide or protein to another polypeptide or protein. In the context of the invention, the peptide linker connects the anchoring polypeptide or protein and the tumor antigen(s). In a particular embodiment, the peptide linker is G4S, composed of 4 glycines and a serine (GGGGS). The G4S linker is commonly used in protein engineering because of its flexibility and resistance to proteases.

The vector(s) may also comprise a nucleic sequence encoding a protein tag (or tag), such as for example the c-myc tag. Thus, the fusion protein may comprise a tag at its end. The tag is used to enable detection of the antigen(s) expressed at the wall of the yeasts.

In a particular embodiment, the yeast is chosen from the genus Saccharomyces, Schizosaccharomyces, Kluveromyces, Ogataea or Candida. Advantageously, the yeast is chosen from the genus Saccharomyces, preferably Saccharomyces cerevisiae. Saccharomyces cerevisiae is particularly advantageous because of the numerous studies showing its safety for humans or animals. The yeast INVSC1 (Life Technologies) is one example thereof. Saccharomyces cerevisiae is also well known for its tolerance when administered in humans or animals. Saccharomyces cerevisiae is especially used in the treatment of rhinitis or chronic rhinopharyngitis, in combination with sulfur and vitamins, the purpose of which is to reduce the inflammation of the mucous membranes of the nose and throat. This yeast is also used as a production system, for example for vaccines, such as hepatitis B vaccine.

The anchoring polypeptide or protein makes it possible to maintain the fusion protein (Ia) at the wall of the genetically modified yeast. Advantageously, the anchoring polypeptide or protein is a protein or a polypeptide expressed naturally at the wall of the yeasts. The anchoring polypeptide or protein may be chosen from a protein or polypeptide expressed naturally by the chosen yeast species or an exogenous protein or polypeptide, i.e., a polypeptide or protein not expressed by the chosen yeast species, for example a polypeptide or protein expressed naturally by a yeast species other than the yeast species chosen to be genetically modified. Advantageously, the anchoring polypeptide or protein is a yeast polypeptide or protein chosen from Aga2p, Sed1p, Cwp1p, Cwp2p, Flo1p C, Tip1p or Tir1p/Srp1p. In a preferred embodiment, the anchoring polypeptide or protein is chosen from Aga2p or Sed1p. The nucleic sequence encoding Aga2p is SEQ ID No: 10 and the nucleic sequence encoding Sed1p is SEQ ID No: 11.

When the Aga2p polypeptide is used in the fusion protein (Ia), it is necessary for the genetically modified yeast to also express Aga1p. Indeed, Aga1p is integrated into the wall of the yeast and Aga2p binds to Aga1p via a disulfide bridge (see FIG. 1 and FIG. 2). The yeast chosen to be genetically modified may express Aga1p naturally. Nevertheless, when the anchoring polypeptide or protein is Aga2p, the process according to the invention preferably comprises the introduction, into the yeast, of a nucleic sequence encoding Aga1p. This nucleic sequence encoding Aga1p may be borne by a vector of step a1) or by another vector which is also introduced into the yeast. The nucleic sequence encoding Aga1p is SEQ ID No: 9.

In a particular embodiment, the vector(s) used in step a) further comprise(s) a nucleic sequence encoding a protein or a polypeptide for targeting dendritic cells. Thus, in this embodiment, the nucleic sequence (IIa) or (IIb) comprises a nucleic sequence encoding a polypeptide or protein for targeting dendritic cells (i.e. n=1 for the nucleic sequence encoding a polypeptide or protein for targeting dendritic cells).

“Protein or polypeptide for targeting dendritic cells” is intended to mean any molecule that is peptide in nature and enables the immunotherapeutic yeast of the invention and human or animal dendritic cells to be brought together. This bringing together enables the dendritic cells to internalize the immunotherapeutic yeast and therefore to internalize the tumor antigen(s) expressed at the wall of said yeast. In other words, the targeting polypeptide or protein makes it possible to facilitate the interaction between the immunotherapeutic yeast and the human or animal dendritic cells. This interaction facilitates the endocytosis of the tumor antigen(s) and consequently the presentation of this or these tumor antigen(s) by the dendritic cells to the T lymphocytes.

The proteins or polypeptides for targeting dendritic cells include all proteins or polypeptides able to specifically bind a dendritic cell endocytic receptor, for example the receptor DEC205 (CD205).

In a particular embodiment, the polypeptide or the protein for targeting the dendritic cells is chosen from:
an antibody or antibody fragment directed against (i.e. capable of binding specifically to) the receptor DEC205 (CD205); or
the plasminogen activator (PLA) of the bacterium Yersinia pestis, or a sequence derived from the plasminogen activator (PLA) of the bacterium Yersinia pestis. PLA is known to bind naturally to CD205.

An antibody fragment may be chosen from the fragments Fv, Fab, Fab′, Fab′-SH and F(ab′)₂; diabodies; linear antibodies; antibodies with a single chain (e.g. scFv); preferably, an antibody fragment according to the invention is an scFv, for example an scFv directed against CD205 of sequence SEQ ID No: 13.

The protein CD205 is a 205 kDa integral membrane glycoprotein, homologous to the macrophage mannose receptor and related receptors. CD205 is an endocytic multilectin receptor that is used by dendritic cells and by epithelial cells of the thymus to direct antigens captured in extracellular spaces to a specialized antigen processing compartment. The plasminogen activator PLA from Yersinia pestis is a protease that plays an important role in the progression of the bacteria. It has been shown that Yersinia pestis was able to use the receptor DEC205 via the plasminogen activator PLA in order to disseminate itself in mice (J Biol Chem. 2008 Nov. 14; 283 (46): 31511-21).

In a particular embodiment, the plasminogen activator (PLA) may be the corresponding whole sequence or a partial sequence or a mutated whole sequence or else a mutated partial sequence. The partial and/or mutated PLA sequences are also referred to as PLA-derived sequences.

In a particular embodiment, the tumor antigen(s) expressed at the wall of the immunotherapeutic yeasts are chosen from antigens of solid or liquid tumors, preferably from antigens of solid tumors. Advantageously, the tumor antigen(s) is/are chosen from Melan-A, Tyrosinase, gp100, MAGE-B1, MAGEA1, MAGEA10, MAGEA11, MAGEA12, MAGEA2, MAGEA2B, MAGEA3, MAGEA4, MAGEA6, MAGEA8, MAGEA9, MAGEB1; MAGEB10, MAGEB16, MAGEB18, MAGEB2, MAGEB3, MAGEB4, MAGEB5, MAGEB6, MAGEB6B, New York Esophageal 1 antigen (NY-ESO-1), MAGEC1, MAGEC2, L-antigen (LAGE), TRP-1, TRP-2, P53, KRAS, CEA, WT1, MUC1, SART3, SURVIVIN 2B, RNF43/TOMM34, TGFBRII, HER2/neu, BRAF, PI3K, APC, BAX, beta-2 microglobulin, telomerase or NRAS.

### Immunotherapeutic Yeast

Another subject of the invention is an immunotherapeutic yeast able to be obtained by carrying out the process according to the invention.

Another subject of the invention is a genetically modified and permeabilized immunotherapeutic yeast which expresses, at its wall:
(i) one or more tumor antigen(s), preferably chosen from Melan-A, Tyrosinase, gp100, MAGE-B1, MAGEA1, MAGEA10, MAGEA11, MAGEA12, MAGEA2, MAGEA2B, MAGEA3, MAGEA4, MAGEA6, MAGEA8, MAGEA9, MAGEB1; MAGEB10, MAGEB16, MAGEB18, MAGEB2, MAGEB3, MAGEB4, MAGEB5, MAGEB6, MAGEB6B, New York Esophageal 1 antigen (NY-ESO-1), MAGEC1, MAGEC2, L-antigen (LAGE), TRP-1, TRP-2, P53, KRAS, CEA, WT1, MUC1, SART3, SURVIVIN 2B, RNF43/TOMM34, TGFBRII, HER2/neu, BRAF, PI3K, APC, BAX, beta-2 microglobulin, telomerase or NRAS; and
(ii) optionally a polypeptide or protein for targeting dendritic cells.

Preferably, the immunotherapeutic yeast according to the invention is genetically modified, permeabilized and inactivated.

In a particular embodiment, the yeast of the invention expresses, at its wall, a fusion protein of formula (Ia):

[protein or polypeptide for targeting dendritic cells]ₙ-[tumor antigen]ₓ-[peptide linker]ₘ-[polypeptide or protein for anchoring to the wall of the genetically modified yeast] (Ia);

n is equal to 0 or 1, m is equal to 0 or 1, and x is an integer ranging from 1 to 300, preferably ranging from 1 to 50, preferably ranging from 1 to 9, preferably ranging from 1 to 5, for example from 1 to 4, from 1 to 3, from 1 to 2, for example equal to 1, equal to 2, equal to 3, equal to 4, equal to 5.

In a particular embodiment, the agent for targeting dendritic cells is chosen from an antibody able to specifically bind to the protein DEC-205, a fragment of antibody able to specifically bind to the protein DEC-205, the plasminogen activator (PLA) of the bacterium Yersinia pestis a sequence derived from the plasminogen activator (PLA) of the bacterium Yersinia pestis. The agent for targeting dendritic cells is described above.

As described above:
when x is an integer greater than 1, that is to say when x is an integer ranging from 2 to 300, the tumor antigens may be identical or different. For example, the tumor antigens may all be different.
when x is an integer greater than 1, that is to say when x is an integer ranging from 2 to 300, the tumor antigens may be separated or not by a linker peptide; and/or
when x is an integer greater than 1, that is to say when x is an integer ranging from 2 to 300, the tumor antigens may be identical or different. For example, when x=2, the tumor antigens may be two MAGEA1 antigens or one MAGEA1 antigen and one MAGEA2 antigen. In this embodiment, the tumor antigens may be placed one after the other or separated by a peptide sequence that connects said tumor antigens to one another.

### Immunotherapeutic Composition

Another subject of the invention is an immunotherapeutic composition comprising a yeast as described above and a pharmaceutically acceptable carrier.

“Immunotherapeutic composition” is intended to mean a composition intended for the prevention or treatment of a disease, after administration to humans or animals, said composition comprising one or more components capable of stimulating a humoral immune response and/or a cell-mediated immune response. Included among the components capable of stimulating a humoral immune response and/or a cell-mediated immune response, are tumor antigens, microorganisms such as viruses, bacteria or yeasts, but also cell lysates, dendritic cells, genetically modified cytotoxic lymphocytes, cytokines, immune system checkpoint inhibitors.

More particularly, the immunotherapeutic composition according to the invention is intended for the prevention or treatment of a cancer. In particular, the immunotherapeutic composition is capable of stimulating a cell-mediated immune response. The cell-mediated immune response is characterized by the intervention of cells of the immune system developing direct cytotoxicity, that is to say cells capable of destroying target cells expressing a non-self antigen. Cells of the immune system capable of cytotoxicity are represented by NK (Natural Killer) cells and cytotoxic T lymphocytes. Cytotoxic T lymphocytes are a subset of T lymphocytes, capable of inducing the death by apoptosis of cells infected with an infectious agent or of inducing the death of cancer cells.

In order to give rise to an immune response, the antigen must be presented to the CD8+ T lymphocytes (cytolytic response) or CD4+ T lymphocytes (auxiliary response) by a molecule of the major histocompatibility complex (MHC) of class I or II, respectively, borne by an antigen-presenting cell. Among the antigen-presenting cells, dendritic cells have the best performance: they have the ability not only to activate naive T lymphocytes but also to induce a cytolytic cellular and humoral response by the presentation of antigens in the context of MHC class I or II molecules. The steps of catabolism of the antigen are strictly correlated with the stages of dendritic cell maturation. Only immature dendritic cells, concentrated in peripheral tissues, can phagocytize and cause endocytosis of soluble and particulate antigens, but they cannot, at this stage, present them to T lymphocytes because the MHC molecules are retained in their lysosomes. On the other hand, the process of maturation of these dendritic cells, controlled by inflammatory signals and by the CD40-L/CD40 molecule pair, is accompanied by morphological changes, by the relocation of MHC class I and II molecules to the membrane, by the expression of lymphocyte co-stimulatory molecules, and above all by the migration of the dendritic cells from the peripheral tissues to the ganglia and the spleen. In these organs, the mature dendritic cells are able to present, to the T lymphocytes, the antigen proteolyzed into peptides and complexed to the MHC-I molecules.

The immunotherapeutic yeast contained in the immunotherapeutic composition is capable of interacting with dendritic cells and of stimulating a cell-mediated immune response by the activation of T lymphocytes. The immune response thus results in a physiological response reflected by a regression in the size of the targeted tumor. In vivo, in animals, the size of a tumor is measured in cubic millimeters (mm³) and the regression in the size of a tumor treated by a therapeutic means is commonly evaluated as a percentage relative to the size of an untreated tumor. In a human patient, the regression in the size of a tumor is measured as a percentage relative to the initially detected tumor before treatment.

“Pharmaceutically acceptable carrier”, also referred to as “excipient”, is intended to mean any component other than the active ingredient(s) which is present in a medicament or used for the manufacture thereof. The function of an excipient is to transport the active ingredient(s), contributing in this way to certain properties of the product such as stability, biopharmaceutical profile, appearance, acceptability to the patient and/or ease of manufacture. The formulation of an immunotherapeutic composition generally comprises several excipients. This includes hydrophilic excipients such as water (purified or for injection), alcohols (ethanol, glycols, glycerol or polyethylene glycols), gelling agents such as gums, substances extracted from algae, proteins, cellulose and derivatives thereof and synthetic gelling agents. This also includes lipophilic excipients such as glycerides of natural or semi-synthetic origin and non-glycerol lipophilic excipients such as fatty acids, fatty alcohols, hydrocarbons and silicones. This also includes emulsifying excipients, including ionic, anionic, cationic or amphoteric surfactants and nonionic surfactants. Yet other components may serve as excipients, such as sugars (sucrose, glucose, fructose, lactose, sorbitol, starch) or mineral products such as colloidal silicas, talc, kaolin or else titanium oxide.

In a particular embodiment, the immunotherapeutic composition of the invention also comprises a therapeutic agent. Advantageously, the therapeutic agent is chosen from an anticancer polypeptide or a chemotherapy agent. It may also be polysaccharides, lipid derivatives, vitamins, nucleic acids or aptamers.

The anticancer polypeptide may be chosen from cytokines, chemokines, hormones, antibodies, antibody fragments, agonists, antagonists or growth factors. This list is not exhaustive.

Chemotherapy agents are well known to those skilled in the art. They are grouped into several families, which are alkylating agents, spindle agents, spindle poisons (vinca alkaloids and related), spindle stabilizers (taxanes), anti-metabolites, proteasome inhibitors or topoisomerase inhibitors. Advantageously, the chemotherapy agent is chosen from cyclophosphamide, docetaxel (taxane family), doxorubicin (anthracycline family), epirubicin (anthracycline family), fluorouracil (also referred to as 5-FU), methotrexate, paclitaxel (taxane family), anthracyclines, capecitabine, eribulin, gemcitabine or vinorelbine. This list is not exhaustive.

Another subject of the invention is an immunotherapeutic yeast according to the invention or an immunotherapeutic composition according to the invention for use thereof as medicament, more particularly for use thereof in the treatment or prevention of cancer.

“Cancer” is intended to mean a large group of diseases that can affect any part of the body, one of the common features of which is the rapid and uncontrolled proliferation of abnormal cells that can spread into other organs, forming what are referred to as metastases. The term “cancer” covers solid cancers and liquid cancers, also referred to as hematopoietic cancers, that include leukemias and lymphomas. In a particular embodiment, the cancer is a solid cancer. Solid cancers can develop in any tissue. A distinction is made between carcinomas and sarcomas. In a preferred embodiment, the cancer is chosen from melanomas, squamous cell carcinomas, breast cancers, carcinomas of the head and neck, thyroid carcinomas, soft tissue sarcomas, bone sarcomas, testicular cancers, prostate cancers, ovarian cancers, bladder cancers, skin cancers, brain cancers, angiosarcomas, hemangiosarcomas, mastoid cell tumors, liver cancers, lung cancers, pancreatic cancers, gastrointestinal cancers, renal cell carcinomas and all metastatic cancers that derive from this list. In a preferred embodiment of the invention, the solid cancer is a melanoma, whether superficial spreading melanoma, nodular melanoma, Dubreuilh melanoma or acral lentiginous melanoma and the metastasized forms that may be associated therewith. In another preferred embodiment of the invention, the solid cancer is a colon cancer. Advantageously, when the solid cancer is a melanoma, the immunotherapeutic yeast expresses, at its wall, one or more antigen(s) chosen from Melan-A, Tyrosinase, gp100, MAGE-B1, MAGEA1, MAGEA10, MAGEA11, MAGEA12, MAGEA2, MAGEA2B, MAGEA3, MAGEA4, MAGEA6, MAGEA8, MAGEA9, MAGEB1, MAGEB10, MAGEB16, MAGEB18, MAGEB2, MAGEB3, MAGEB4, MAGEB5, MAGEB6, MAGEB6B, New York Esophageal 1 antigen (NY-ESO-1), MAGEC1, MAGEC2, L-antigen (LAGE), TRP-1 or TRP-2. When the solid cancer is a colon cancer, the immunotherapeutic yeast advantageously expresses, at its wall, one or more antigen(s) chosen from P53, KRAS, CEA, WT1, MUC1, SART3, SURVIVIN 2B, RNF43/TOMM34, TGFBRII, HER2/neu, BRAF, PI3K, APC, BAX, beta-2 microglobulin, telomerase or NRAS.

The immunotherapeutic yeast or the immunotherapeutic composition according to the invention may be administered via injections, intramuscularly, intraperitoneally, intravenously or else subcutaneously, orally or by respiratory/pulmonary route. Depending on the mode of administration, the dosage form will be adapted. Those skilled in the art know how to adapt the dosage forms that lend themselves to the chosen route of administration. For example, for oral administration, the dosage form may be chosen from tablets, including orodispersible tablets, capsules, gel capsules, oral solutions. For pulmonary administration, the dosage form may be in the form of a spray or products for inhalation. Advantageously, the immunotherapeutic yeast or the immunotherapeutic composition is administered by subcutaneous injection. In a particular embodiment, the yeast or the immunotherapeutic composition according to the invention is administered to humans or animals at a rate of one or more doses per week, or of one or more doses per month, said dose ranging from 0.1 to 200 YU (Yeast Unit), preferably 0.1 to 2 YU, or 0.1 to 5 YU, or 0.1 to 10 YU, or 1 to 10 YU, 10 to 20 YU, 20 to 30 YU, 30 to 40 YU, 40 to 50 YU, or 50 and 100 YU, 100 and 150 YU, or 150 YU and 200 YU, with one YU equal to 10⁷ yeasts.

### DESCRIPTION OF THE FIGURES

FIG. 1 is a diagram illustrating a yeast expressing, at its wall, the polypeptide MEQLESIINFEKLTEWTSA (SEQ ID NO: 14) representing the OVA1 antigen derived from ovalbumin, said antigen being expressed at the wall of the yeast via the anchoring polypeptide Aga2p linked to Aga1p via a disulfide bridge. The OVA1 antigen is bonded to the anchoring polypeptide via a G4S linker, composed of 4 glycines and a serine. C-myc is a tag that makes it possible to detect and confirm the expression of the OVA1 antigen at the wall of the genetically modified yeast.

FIG. 2 is a diagram illustrating a yeast expressing, at its wall, the polypeptide MEQLESIINFEKLTEWTSA (SEQ ID NO: 14) representing the OVA1 antigen derived from ovalbumin, said antigen being expressed at the wall of the yeast via an anchoring protein, Sed1p. The OVA1 antigen is bonded to the anchoring polypeptide via a G4S linker, composed of 4 glycines and a serine. C-myc is a tag that makes it possible to detect and confirm the expression of the OVA1 antigen at the wall of the genetically modified yeast.

FIG. 3 is a diagram illustrating a yeast expressing, at its wall, the polypeptide MEQLESIINFEKLTEWTSA (SEQ ID NO: 14) representing the OVA1 antigen derived from ovalbumin, said antigen being expressed at the wall of the yeast via the anchoring polypeptide Aga2p linked to Aga1p via a disulfide bridge. The OVA1 antigen is attached to the anchoring polypeptide via a G4S linker, composed of 4 glycines and a serine. The yeast also expresses, at its wall, the ScFv fragment of an antibody directed against the DEC-205 receptor (ScFv anti-DEC205). C-myc is a tag that makes it possible to detect and confirm the expression of the OVA1 antigen at the wall of the yeast.

FIG. 4 is a diagram illustrating a yeast expressing, at its wall, the polypeptide MEQLESIINFEKLTEWTSA (SEQ ID NO: 14) representing the OVA1 antigen derived from ovalbumin, said antigen being expressed at the wall of the yeast via an anchoring protein, Sed1p. The OVA1 antigen is bonded to the anchoring polypeptide via a G4S linker, composed of 4 glycines and a serine. The yeast also expresses, at its wall, the ScFv fragment of an antibody directed against the DEC-205 receptor (ScFv anti-DEC205). C-myc is a tag that makes it possible to detect and confirm the expression of the OVA1 antigen at the wall of the yeast.

FIG. 5 is a diagram illustrating the assembly of a vector with nucleic sequences by the Golden Gate method.

FIG. 6 is a diagram illustrating a vector assembled by the Golden Gate method.

FIG. 7 is a flow cytometry spectrum which shows expression of the OVA1 peptide at the wall of the yeasts using an antibody directed against the c-myc tag.

FIG. 8 is a diagram which shows the activation of cytotoxic CD8+ T lymphocytes after cross-presentation of the antigen SIINFEKL (OVA 257-264) by the dendritic cells to said cytotoxic CD8+ T lymphocytes, with increasing doses of antigen SIINFEKL (OVA 257-264) (from 0 nM to 10 nM). The antigen SIINFEKL is free, that is to say not attached to the wall of the yeasts, and wild-type yeasts are used as an adjuvant with a MOI (Multiplicity of infection) of 20.

FIG. 9 is a diagram which shows the activation of cytotoxic CD8+ T lymphocytes after cross-presentation by dendritic cells using permeabilized or non-permeabilized yeasts. A) non-permeabilized (dots) and permeabilized (lines) wild-type yeasts, B) yeasts expressing the OVA1 antigen in the cytosol, non-permeabilized (dots) and permeabilized (lines), C) yeasts expressing OVA1 at the wall via the anchoring protein Sed1p, non-permeabilized (dots) and permeabilized (lines), D) yeasts expressing the OVA1 antigen and an anti-DEC205 ScFv fragment at the wall using the anchoring protein Sed1p, E) yeasts expressing the OVA1 antigen and an anti-DEC205 ScFv fragment at the wall via the anchoring polypeptide Aga2p, with an integrative plasmid.

FIG. 10 is a graph showing tumor growth in cubic millimeters (mm³) in mice after tumor challenge on D₀ with 5×10⁵ B16-OVA (MO5) melanoma cells injected subcutaneously. The wild-type mice (WT for Wild Type) are represented by circles. The mice treated with permeabilized yeasts expressing OVA1 fused to anti-DEC-205 scFv at their wall via the anchoring protein Sed1p are represented by triangles. A dose of 1 YU (1×10⁷ yeasts) was injected three times, seven days before the intraperitoneal tumor challenge, three days before the subcutaneous tumor challenge, then three days after the subcutaneous tumor challenge.

FIG. 11 is a graph showing the survival rate of the mice after the same tumor challenge described for FIG. 10. The wild-type mice (WT for Wild Type) are represented by circles.

The mice treated with permeabilized yeasts expressing OVA1 fused to anti-DEC-205 scFv at their wall via the anchoring protein Sed1p are represented by triangles.

FIG. 12 is a diagram illustrating a yeast expressing, at its wall, the polypeptide SPSYAYHQF (SEQ ID NO: 15) representing the AH1A5 antigen derived from AH-1, said antigen being expressed at the wall of the yeast via an anchoring protein, Sed1p. The AH1A5 antigen is bonded to the anchoring polypeptide via a G4S linker, composed of 4 glycines and a serine. C-myc is a tag that makes it possible to detect and confirm the expression of the AH1A5 antigen at the wall of the genetically modified yeast. The yeast also expresses, at its wall, the ScFv fragment of an antibody directed against the DEC-205 receptor (ScFv anti-DEC205 or ScFv DEC205).

FIG. 13 is a diagram illustrating a yeast expressing, at its wall, the polypeptide TAPDNLGYM (SEQ ID NO: 16) representing the TRP1 antigen, said antigen being expressed at the wall of the yeast via an anchoring protein, Sed1p. The TRP1 antigen is bonded to the anchoring polypeptide via a G4S linker, composed of 4 glycines and a serine. C-myc is a tag that makes it possible to detect and confirm the expression of the TRP1 antigen at the wall of the genetically modified yeast. The yeast also expresses, at its wall, the ScFv fragment of an antibody directed against the DEC-205 receptor (ScFv anti-DEC205 or ScFv DEC205).

FIG. 14 is a diagram illustrating a yeast expressing, at its wall, the polypeptide SVYDFFVWL (SEQ ID NO: 17) representing the TRP2 antigen, said antigen being expressed at the wall of the yeast via an anchoring protein, Sed1p. The TRP2 antigen is bonded to the anchoring polypeptide via a G4S linker, composed of 4 glycines and a serine. C-myc is a tag that makes it possible to detect and confirm the expression of the TRP2 antigen at the wall of the genetically modified yeast. The yeast also expresses, at its wall, the ScFv fragment of an antibody directed against the DEC-205 receptor (ScFv anti-DEC205 or ScFv DEC205).

FIG. 15 is a diagram illustrating a yeast expressing, at its wall, the polypeptides TAPDNLGYM (SEQ ID NO: 16), MEQLESIINFEKLTEWTSA (SEQ ID NO: 14) and SVYDFFVWL (SEQ ID NO: 17) representing, respectively, the TRP1, OVA1 and TRP2 antigen, said antigens being expressed at the wall of the yeast via an anchoring protein, Sed1p. The antigens are bonded to the anchoring polypeptide via a G4S linker, composed of 4 glycines and a serine, and also separated from one another by a G4S linker. C-myc is a tag that makes it possible to detect and confirm the expression of the antigens at the wall of the genetically modified yeast. The yeast also expresses, at its wall, the ScFv fragment of an antibody directed against the DEC-205 receptor (ScFv anti-DEC205 or ScFv DEC205).

FIG. 16 is a flow cytometry spectrum with an anti-C-myc antibody of yeasts expressing, at their walls, the antigens AH1A5 (FIG. 16A), TRP1 (FIG. 16B), TRP2 (FIG. 16C) or OVA1-TRP1-TRP2 (FIG. 16D).

FIG. 17 is a diagram which shows the absorbance at 405 nm of yeasts treated in different ways and brought into contact with para-nitrophenyl phosphate (pNPP). pNPP is the substrate of an enzyme, alkaline phosphatase, which is naturally present in the cytosol of the yeast. pNPP does not penetrate into the cytosol of a non-permeabilized yeast. On the other hand, when the yeast is permeabilized, the pNPP can enter the yeast and be hydrolyzed by the alkaline phosphatase present in the cytosol. The hydrolyzed pNPP then assumes a yellow color which can be read by the absorbance at 405 nm.

FIG. 18 is a diagram which shows the absorbance at 405 nm of yeasts treated in different ways and brought into contact with para-nitrophenyl phosphate (pNPP). pNPP is the substrate of an enzyme, alkaline phosphatase, which is naturally present in the cytosol of the yeast. pNPP does not penetrate into the cytosol of a non-permeabilized yeast. On the other hand, when the yeast is permeabilized, the pNPP can enter the yeast and be hydrolyzed by the alkaline phosphatase present in the cytosol. The hydrolyzed pNPP then assumes a yellow color which can be read by the absorbance at 405 nm.

FIG. 19 is a diagram which shows the activation of cytotoxic CD8+ T lymphocytes after cross-presentation of yeasts expressing, at their wall, the antigen MEQLESIINFEKLTEWTSA (SEQ ID NO: 14) in the presence of dendritic cells. The yeasts were treated in different ways to obtain permeabilized or non-permeabilized cells.

The examples given below present embodiments of the invention; these examples are given by way of illustration and in no way limit the scope of the claims.

### EXAMPLES

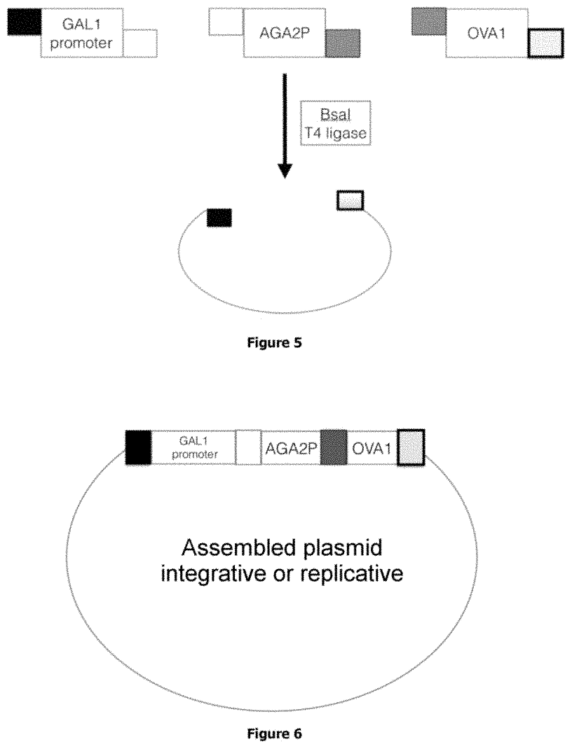

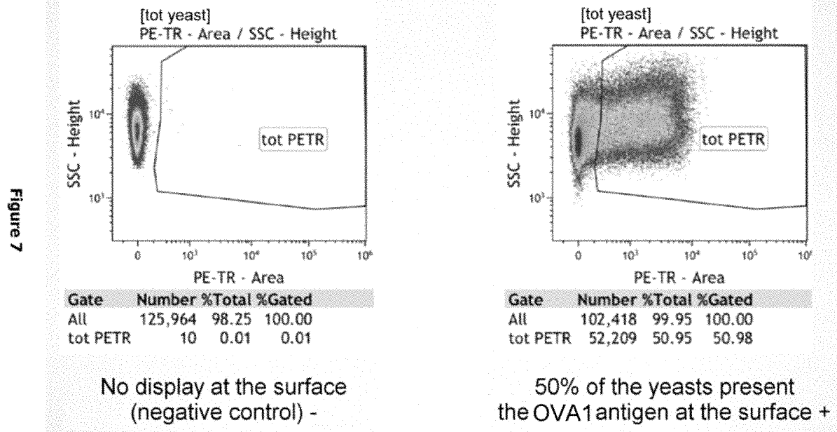

### Example 1: Construction of the Plasmids Introduced Into the Yeasts (FIGS. 5 and 6)

Two types of plasmids were created by Abolis Biotechnologies (iSSB, Génopole, Evry, France), an integrative plasmid and a replicative plasmid.

For the replicative plasmid, an episomal plasmid fragment containing the 2 micron yeast origin of replication, the selection marker URA3 (SEQ ID No. 4) and ampicillin resistance were assembled by the Gibson method (Gibson Assembly Master Mix, NEB, Inc.) then the plasmid was transformed with the inserts by Golden Gate according to the process described in FIGS. 5 and 6.

For the integrative plasmids, two plasmids containing sites for homologous recombination with the genome of the genetically modified yeast, the selection markers URA3 (SEQ ID No. 4) and TRP1 (SEQ ID No. 8), and also the kanamycin resistance gene were transformed by Golden Gate according to the process illustrated in FIGS. 5 and 6.

These two plasmids were assembled by Golden Gate from the following fragments:
the inducible promoter of the yeast gene GAL1, synthesized from the sequence present on chromosome II of Saccharomyces cerevisiae (SEQ ID NO: 1).
the terminator of the gene CYC1, a gene terminator widely used in the scientific community, extracted from a “BioBrick” from the “Parts iGEM Registry” (SEQ ID NO: 2). It makes it possible to indicate to a polymerase the end of the transcription of the gene which encodes the recombinant protein expressed in the genetically modified yeast.
the synthetic terminator T27, placed upstream of the CYC1 terminator to increase production of the recombinant proteins (SEQ ID NO: 3) was synthesized from the sequence of the terminator designated T27 in the article “Short Synthetic Terminators for Improved Heterologous Gene Expression in Yeast, Curran et al., ACS Synth Bio. 2015 Jul. 17; 4 (7): 824-32”. It reinforces the action of the CYC1 terminator.
the gene URA3 was extracted from the yeast genome with its promoter and its terminator (SEQ ID NO: 4)
the kanamycin resistance antibiotic (Kan) and the bacterial origin of replication pMB1 originates from the plasmid pSB1K3 (SEQ ID NO: 5)
the fluorescent yellow reporter gene YFP No. BBa_E0030 with the promoter pLAC No. BBa_R0010 and terminator No. BBa_B0015, all originate from the catalog “parts iGEM registry”.
the insertion sites, named XI-2 UP (SEQ ID NO: 6) and XI-2 DOWN (SEQ ID NO: 7) in the article by Mikkelsen et al, 2012 were amplified from the genome of the yeast.

The nucleotide sequences named “insertion sites” enable the chromosomal integration of the recombinant nucleotide sequences by homologous recombination in the genetically modified yeast (Microbial production of indolylglucosinolate through engineering of a multi-gene pathway in a versatile yeast expression platform, Metab Eng. 2012 March; 14 (2): 104-11), Mikkelsen M D, Buron LD, Salomonsen B, Olsen C E, Hansen B G, Mortensen U H, Halkier B A.)

For Golden Gate insertion of the nucleic sequences encoding the fusion proteins in the plasmids, the nucleic sequences were synthesized to contain BsaI cleavage sites and end-complementary primers. An example of pairs of primers used in the implementation of the invention is: 5′ of the insert, GGTCTCTAATG (SEQ ID NO: 18) and 3′ of the insert, GAGTTGAGACC (SEQ ID NO: 19). These primers are only compatible with the pieces that are inserted before and after, such that all the fragments are assembled in the correct order by mixing them in a single ligation reaction (FIG. 5 and FIG. 6). The insert is composed for example of
the nucleic sequence encoding an anchoring and addressing polypeptide Aga2p, the nucleic sequence encoding the OVA1 peptide (tumor antigen model) and the sequence encoding c-myc,
the nucleic sequence encoding an anchoring polypeptide Sed1p, the nucleic sequence encoding the peptide AH1-A5 (colon cancer antigen), the sequence encoding c-myc, the sequence encoding the targeting polypeptide ScFv DEC-205 and the nucleic sequence encoding an addressing polypeptide derived from the yeast pheromone protein Mf(alpha)1p and named “Pre pro alpha factor leader peptide”,
the nucleic sequence encoding an anchoring polypeptide Sed1p, the nucleic sequence encoding the peptide TRP1 (melanoma antigen), the sequence encoding c-myc, the sequence encoding the targeting polypeptide ScFv DEC-205 and the nucleic sequence encoding an addressing polypeptide derived from the yeast pheromone protein Mf(alpha)1p and named “Pre pro alpha factor leader peptide”,
the nucleic sequence encoding an anchoring polypeptide Sed1p, the nucleic sequence encoding the peptide TRP2 (melanoma antigen), the sequence encoding c-myc, the sequence encoding the targeting polypeptide ScFv DEC-205 and the nucleic sequence encoding an addressing polypeptide derived from the yeast pheromone protein Mf(alpha)1p and named “Pre pro alpha factor leader peptide”,
the nucleic sequence encoding an anchoring polypeptide Sed1p, the nucleic sequence encoding the peptides OVA1, TRP1 and TRP2, the sequence encoding c-myc, the sequence encoding the targeting polypeptide ScFv DEC-205 and the nucleic sequence encoding an addressing polypeptide derived from the yeast pheromone protein Mf(alpha)1p and named “Pre pro alpha factor leader peptide”,
the nucleic sequence encoding an addressing polypeptide derived from the yeast pheromone protein Mf(alpha)1p and named “Pre pro alpha factor leader peptide”, the nucleic sequence encoding the peptide OVA1 and c-myc, and the nucleic sequence encoding the anchoring protein Sed1p.

The method of assembly by Golden Gate was as follows:

1 μl of T4 DNA ligase concentrated to 400 000 units per ml was mixed with 2 μl of T4 DNA ligase 10× buffer, 1 μl of high-fidelity restriction enzyme BsaI concentrated to 20 000 units per ml, 50 ng of each nucleotide sequence inserted (sequences from 1 to 8) and also 50 ng of the plasmid receiving the nucleotide insert. The reaction medium was made up to 25 μl with deionized water. The reaction medium was then subjected to different temperature cycles to enable the enzymatic reaction of cleavage by the enzyme BsaI (cycles at 37° C.) and the ligation of the digested nucleotide sequences and also of the plasmid digested by the T4 ligase enzyme (cycles at 16° C.) according to this protocol:

1st step: 98° C. for 2 minutes,

2nd step (repeated 32 times):
37° C. for 30 seconds
16° C. for 30 seconds

3rd step: 65° C. for 10 minutes

4th step: 12° C. for 10 minutes

Bacterial transformation was performed with the Golden Gate solution obtained after these temperature cycles. 10 μl of the reaction volume were taken off and brought into contact with 20 μl of E. coli competent bacteria (E. coli DH5-Alpha High Efficiency, NEB, Inc.). The bacteria were streaked on a culture medium containing a suitable selection antibiotic, Kanamycin or Ampicillin. After 24 hours of culture at 37° C., an isolated colony was placed in liquid culture at 37° C. in a medium containing the same selection antibiotic as previously. After 24 hours, 2 ml of bacterial culture were taken off to carry out purification of the plasmids. All the plasmids were checked by a colony PCR and sequenced by the Sanger method to verify the correct assembly.

The integrative plasmids were then digested by the enzyme AvrII to linearize them in order to enable homologous recombination. Subsequently, the yeast S. cerevisiae INVSC1 (Life Technologies SAS) was transformed by the lithium acetate method with these linearized integrative plasmids (Transformation of yeast by lithium acetate/single-stranded carrier DNA/polyethylene glycol method, Methods Enzymol. 2002: 350: 87-96, Gietz et al). Briefly, the yeasts were cultured in YPAD (20 g/l glucose, 10 g/l Yeast Extract, 20 g/l bacto peptone) until Log phase, then collected by centrifugation at 3000 g for 5 minutes, washed with sterile water, and transformed with the following solution: 240 μl of PEG 4000 (50% (w/v)), 36 μl of 1.0 M LiAc, 50 μl of single-stranded salmon sperm DNA (2.0 mg/ml), 34 μl of plasmid to be transformed. The yeasts suspended in this transformation solution were placed in a bath at 42° C. for 25 minutes. After centrifugation at 13 000 g for 1 minute, the yeasts were resuspended in YPAD medium for 1 h, before being streaked on a plate containing a selective medium for the plasmid inserted into the yeast. The chromosomal insertion was then verified by genome extraction of the genetically modified yeasts and insert-specific PCR (Extraction of genomic DNA from yeasts for PCR-based applications, Biotechniques 50: 325-328 (2011), Looke et al). Briefly, 100 μl of yeasts cultured at an optical density OD=0.4 at 600 nm were centrifuged, then resuspended in 100 μl of 200 mM lithium acetate (LiAc) 1% SDS solution, vortexed, and incubated for 5 minutes at 70° C. 300 μl of 96% ethanol were added to precipitate the DNA, the solution was vortexed and centrifuged at 13 000 g for 3 minutes. The supernatant was removed and the pellet was washed with 500 μl of 70% ethanol then resuspended in 100 μl of sterile water. 1 μl of supernatant was then used for the PCRs, using primers specific to each genomic insert.

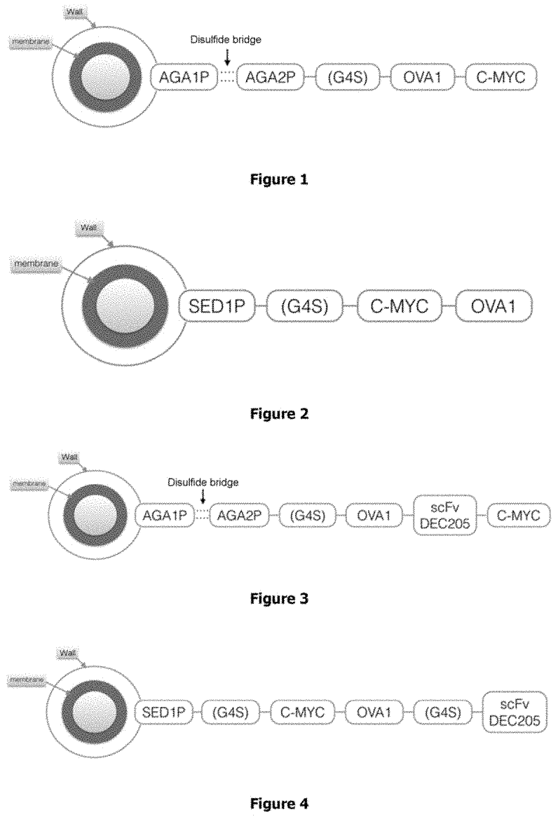

### Example 2: Preparation of the Immunotherapeutic Yeasts (FIGS. 1 to 4)

The yeast Saccharomyces cerevisiae INVSC1 (Life Technologies SAS), a quadruple auxotrophic yeast (URA, TRP, HIS, LEU), was used in all the examples cited. The yeast was genetically modified to express one or more tumor antigen(s) with an anchoring polypeptide or protein, an addressing polypeptide, and a targeting protein or peptide after transformation with the plasmids described in example 1 by the Lithium Acetate method. For the selection of recombinant yeasts, a selective medium without the amino acids URA or TRP was used. The yeast was first cultured at 30° C. until the stationary phase in a selective medium comprising: 20 g/l of glucose, 6.7 g/l of yeast nitrogen base without amino acids, 0.7 g/l of each amino acid from: histidine, leucine and tryptophan. The yeasts were then centrifuged for 5 minutes at 4000 rpm, washed in PBS (Phosphate-Buffered Saline), then resuspended in an inductive selective medium comprising: 20 g/l of galactose, 6.7 g/l of yeast nitrogen base without amino acids, 0.7 g/l of each of the following amino acids from: histidine, leucine and tryptophan. The yeasts were then cultured at 20° C. for 20 h for the induction of the expression of the recombinant proteins by galactose.

For the permeabilization, the yeasts were fixed with 0.5% paraformaldehyde (PFA) for 10 minutes, washed in PBS, then they were treated with a mixture of 50% ethanol, 50% water v/v for 15 minutes and washed again with PBS.

FIG. 1 shows the example of a genetically modified yeast which has been transformed by a first vector containing the nucleic sequence encoding the OVA1 antigen fused to the c-myc tag and the nucleic sequence encoding the anchoring and addressing polypeptide Aga2p (SEQ ID NO: 10). Aga2p was linked via a disulfide bridge to the protein Aga1p (SEQ ID NO: 9) which became anchored in the wall of the yeast and which was produced from a second vector co-transformed with the first vector.

FIG. 2 shows the example of a genetically modified yeast transformed by a single vector containing the OVA1 antigen fused to the c-myc tag and the anchoring protein Sed1p (SEQ ID NO: 11).

FIG. 3 shows the example of a yeast genetically modified by a first vector containing the nucleic sequence encoding the OVA1 antigen fused to the targeting polypeptide ScFv DEC205, the nucleic sequence encoding the c-myc tag and the nucleic sequence encoding the anchoring and addressing polypeptide Aga2p. Aga2p was linked via a disulfide bridge to the protein Aga1p which became anchored in the wall of the yeast and which was produced from a second vector co-transformed in the yeast with the first vector.

FIG. 4 shows the example of a genetically modified yeast which has been transformed by a single vector containing the nucleic sequence encoding the N-terminal addressing polypeptide “prepro alpha factor leader peptide” derived from the yeast pheromone Mf(alpha)1p (SEQ ID NO: 12), the nucleic sequence encoding the targeting polypeptide scFv DEC205 (SEQ ID NO: 13), the nucleic sequence encoding the OVA1 antigen fused to the c-myc tag, and the nucleic sequence encoding the anchoring protein Sed1p.

To enable suitable processing of the antigen by the dendritic cells during cross-presentation assays, the natural flanking sequences of the ovalbumin protein were added to said peptide by synthesis: MEQLESIINFEKLTEWTSA (SEQ ID No. 14). The peptide SIINFEKL combined with the flanking sequences represents OVA1. A G4S linker was placed between the anchoring polypeptide or protein and OVA1. This linker is an amino acid chain composed of 4 glycines and one serine (GGGGS). A protein tag, c-myc, was also added to enable the detection of the complexes at the surface of the yeasts.

The addressing of the fusion protein at the surface of the yeast required an N-terminal secretion signal. For the fusion proteins comprising Aga2p, this protein already contained the required secretion signal. For the fusion proteins using Sed1p at the C-terminus, an additional secretion signal was added at the N-terminus of the fusion protein, using the addressing polypeptide encoded by the “prepro alpha factor leader peptide” nucleic sequence derived from the yeast pheromone Mf(alpha)1p (SEQ ID No. 12). The targeting polypeptide ScFv anti-DEC205 was able to be added among the inserts (FIGS. 3 and 4) (SEQ ID No. 13) to fuse with the OVA1 antigen expressed at the surface using the previously described Golden Gate technique.

### Example 3: Monitoring the Expression of a Peptide at the Surface of the Yeasts (FIG. 7)

The expression of the OVA1 peptide expressed on the surface of the genetically modified yeast was verified by spectral flow cytometry, using yeasts having the same antigen in the cytosol as negative control. After the induction in galactose described in example 2, the yeasts at a concentration of 1×10⁷ cells per ml were suspended in a 2% PFA solution. They were then washed in PBS containing 1% BSA. After washing, they were cultured for 1 hour at room temperature with an anti-c-myc primary antibody at a ratio of 1:100 (Myc.A7, Life Technologies SAS). After 3 washes with PBS containing 1% BSA, they were incubated for 1 h at room temperature with an anti-IgG1 secondary antibody at a ratio of 1:50 (Goat anti-Mouse IgG1, PE-Texas Red, Life Technologies SAS). After washing, they underwent spectral flow cytometry in the PE-Texas red preferential channel.

The results are presented in FIG. 7. This figure shows the expression of the OVA1 peptide at the surface of the yeasts constructed according to the example described in FIG. 2. An anti-c-myc antibody makes it possible to detect the yeasts which are expressing the OVA1 antigen at their surface. In the case in which the yeasts are expressing the antigen in the cytosol, there is no detection by flow cytometry. In the case of the construct of FIG. 2, 50% of the yeasts express OVA1 at their surface.

Conclusion: Spectral flow cytometry demonstrated on the one hand the effective anchoring of the OVA1 antigen to the wall of the yeasts. On the other hand, under the experimental conditions tested, the labeling antibody of the fusion protein was not able to penetrate inside the yeasts, which demonstrated the presentation of the OVA1 antigen on the outside of the yeast.

### Example 4: Measurement of the Activation of Cytotoxic CD8+ T Lymphocytes by Presentation of the Free OVA1 Antigen by the Dendritic Cells in the Presence of Wild-Type Yeasts

This example shows the ability of the OVA1 antigen to activate CD8+ T lymphocytes independently of its expression by a yeast. The antigen was cross-presented to CD8+ T lymphocytes via dendritic cells. The activation of the cytotoxic CD8+ T lymphocytes was measured using a colorimetric test using beta-galactosidase and one of its substrates, CPRG (Chlorophenol red-beta-galactopyranoside).

The test requires a source of dendritic cells, a source of CD8+ T lymphocytes and yeasts modified so as to express an antigen at the surface, said yeasts being non-permeabilized. The dendritic cells used are murine dendritic cells derived from the MutuDC line (obtained from the University of Lausanne). This line originates from immortalized splenic CD8α dendritic cells that retain the ability to cross-present an antigen and to activate killer lymphocytes (KL). The cells of the MutuDC line were cultured in RPMI-1640 medium supplemented with 10% FCS, HEPES, 50 μM 2-mercaptoethanol, 50 U/ml penicillin, and 50 μg/ml streptomycin, at 37° C. with 5% CO₂.

The T lymphocytes used originate from the B3Z hybridoma. The B3Z hybridoma, a line specific for the peptide OVA 257-264 (SIINFEKL), was offered by Institut Curie (Paris V). These cells have the particular feature of producing beta galactosidase under the control of an IL-2 (interleukin 2) promoter. The B3Z cells were cultured in RPMI-1640 medium supplemented with 10% FCS, Glutamax, HEPES, 50 μM 2-mercaptoethanol, 50 U/ml penicillin, and 50 μg/ml streptomycin, at 37° C. with 5% CO₂.

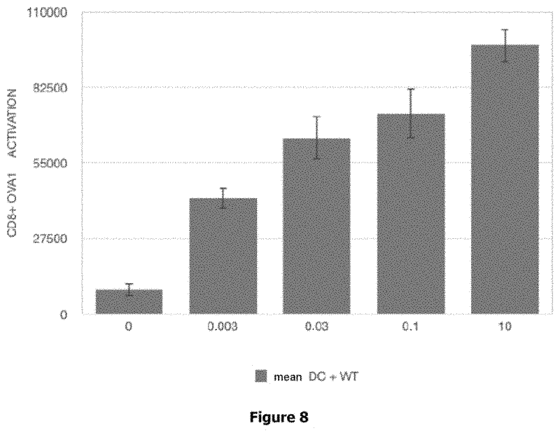

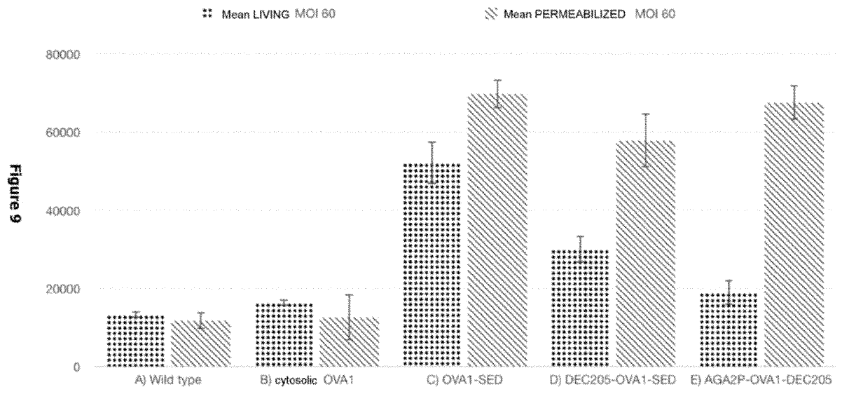

The OVA 257-264 (SIINFEKL) peptide was mixed with the dendritic cells for 5 h in the presence of the wild-type yeasts. The dendritic cells were distributed at an amount of 50 000 cells per well in 96-round-bottom-well plates. The lymphocytes of the B3Z line were subsequently added at an amount of 100 000 per well for 18 h at 37° C. and 5% CO₂. The plates were then washed and the activity of the beta galactosidase produced by the T lymphocytes was measured after addition of 120 μl of lysis buffer which contains PBS, 9 mM MgCl₂, 0.125% NP40 and 0.15 mM chlorophenol red-beta-galactopyranoside). After the color change to red, the fluorescence in the red at 580 nm was read using a ClarioStar plate reader. FIG. 8 shows the ability of the OVA 257-264 (SIINFEKL) antigen to induce activation of the CD8+ T lymphocytes, once cross-presented by dendritic cells. The assay is performed with increasing doses of OVA 257-264 (SIINFEKL) peptide as positive control.

Conclusion: This assay demonstrated that the dendritic cells used, derived from the MutuDC line, were capable of performing the cross-presentation of a tumor antigen in the presence of an adjuvant. This assay also demonstrated the activation of the B3Z cell line of killer lymphocytes in the presence of these dendritic cells. The activation of the B3Z was effected by the production of beta-galactosidase, proportionally to the amount of antigen initially supplied to the dendritic cells.

### Example 5: Measurement of the Activation of Cytotoxic CD8+ T Lymphocytes by Presentation of the OVA1 Antigen by Dendritic Cells, Said OVA1 Antigen Being Expressed at the Wall of the Permeabilized or Non-Permeabilized Yeasts

The experimental conditions of example 4 are applicable to example 5. The condition that differs lies in the use of permeabilized or non-permeabilized genetically modified yeasts to express an antigen at their wall.

The permeabilized or non-permeabilized wild-type yeasts or genetically modified yeasts having the OVA1 antigen in the cytosol or genetically modified yeasts having the OVA1 antigen at their wall were cultured with the cells of the MutuDC line for 5 h at a ratio of 60:1 (MOI 60, Multiplicity of Infection). The lymphocytes of the B3Z line were then added. Permeabilization was performed as previously described by subjecting the yeasts to a treatment by 0.5% PFA for 10 minutes, to washing with PBS, then to a treatment with ethanol (50% ethanol, 50% PBS) for 15 minutes, washing again with PBS, before being co-cultured with the MutuDC. Synergy appeared for the permeabilized yeasts which express the antigen at their surface (FIG. 9), significantly improving the activation of the lymphocytes. As shown in FIG. 9, the genetically modified yeasts having the antigen at their wall induce a stronger activation of the CD8+ T lymphocytes than the yeasts having the antigen in their cytosol. Moreover, the permeabilization had no effect on the yeasts which produce the OVA1 antigen in the cytosol. On the other hand, the permeabilized yeasts which express the OVA1 antigen at their wall significantly improve the activation of the CD8+ T lymphocytes, compared to the non-permeabilized yeasts, with an increase in activation of between 33% and 255% by the permeabilized yeasts compared to the non-permeabilized yeasts.

Conclusion: This cross-presentation assay demonstrated that the genetically modified and permeabilized yeasts very significantly activate killer lymphocytes when these yeasts express the OVA antigen at their wall, compared to non-permeabilized yeasts that also express the OVA antigen at the wall.

### Example 6: Measurement of the Growth of a Melanoma tumor and of the Survival Rate in Mice

The growth of a melanoma was measured on mice receiving permeabilized yeasts having the OVA1 antigen at the wall, coupled to the scFv anti-DEC205 antibody fragment.

Female C57BL/6 mice aged 6 and 8 weeks were maintained and treated in accordance with the Bioethics Committee of the Polish Academy of Sciences.

The melanoma cell line MO5 (B16-OVA, obtained from Pr O. Lantz, Institut Curie, Paris V) was cultured in RPMI Glutamax+10% FCS+50 μM 2-mercaptoethanol Strep/Pen+2 mg/ml G418+60 μg/ml hygromycin B at 37° C. and 5% CO₂. The MO5 line is a B16 melanoma line, transfected with ovalbumin (OVA).

The mice were immunized intraperitoneally 7 days before the tumor challenge, then subcutaneously 3 days before the tumor challenge and 3 days after the tumor challenge, with a single dose of 1 YU at each injection in 100 μl of PBS. During the tumor challenge, the mice received 1×10⁵ MO5 subcutaneously in a volume of 100 μl of PBS. The control is mice immunized by wild-type yeasts (WT). 5 mice were used per experimental group. The tumor volume was evaluated with the formula (a*a*b)/2, where “a” is the shortest tumor axis and “b” the longest tumor axis, in millimeters. Moribund mice whose tumors exceeded 1500 mm³ were sacrificed.

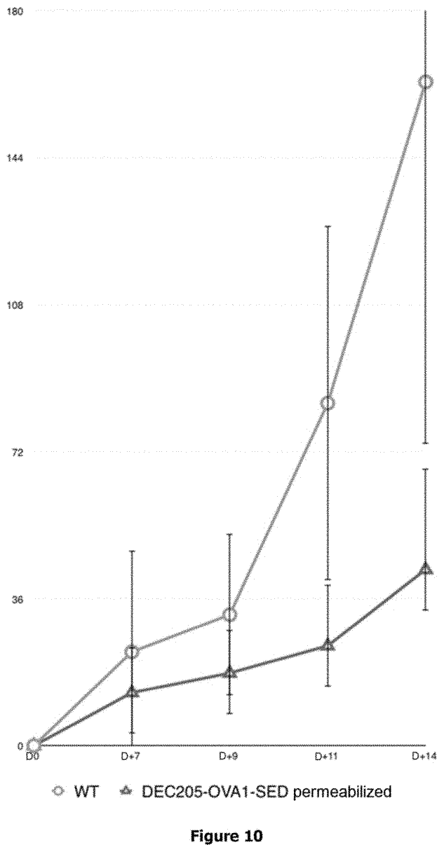

14 days after the tumor challenge, the mice immunized with the permeabilized yeasts expressing DEC205-OVA1-SED had an average tumor volume of 43.1 mm³, versus an average tumor volume of 162.5 mm³ for the mice immunized with the wild-type yeasts (WT), i.e. a tumor volume 3.77 times lower, on average (FIG. 10). The error bars represent the standard deviation of the average tumor volume per experimental group.

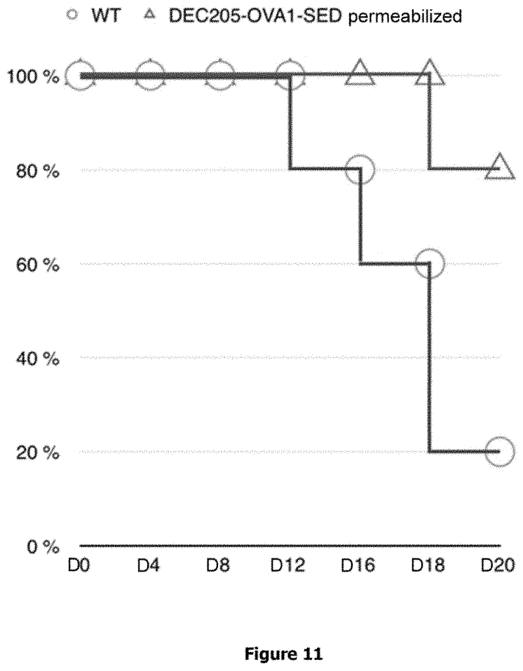

20 days after the tumor challenge, 80% of the mice immunized with permeabilized yeasts expressing DEC205-OVA1-SED had survived, versus 20% of the mice immunized with the wild-type yeasts (WT) (FIG. 11).

Conclusion: Vaccination of the mice with the permeabilized yeasts that have the OVA1 antigen fused to the polypeptide for targeting dendritic cells, scFv DEC205, enabled a significant decrease in tumor growth and a significant increase in the survival of the mice, compared with the mice vaccinated with the wild-type yeasts.

### Example 7: Preparation of Immunotherapeutic Yeasts Expressing, at Their Wall, the Tumor Antigens AH1-A5, TRP1, TRP2 or OVA1-TRP1-TRP2

The yeast Saccharomyces cerevisiae INVSC1 (Life Technologies SAS), a quadruple auxotrophic yeast (URA, TRP, HIS, LEU), was used. The yeast was genetically modified to express one or more tumor antigen(s) (AH1-A5, TRP1, TRP2 or OVA1-TRP1-TRP2) with an anchoring polypeptide, an addressing polypeptide, and a targeting polypeptide after transformation with the plasmids described in example 1 by the Lithium Acetate method.

For the selection of recombinant yeasts, a selective medium without the amino acids URA or TRP was used. The recombinant yeasts were first cultured at 30° C. until the stationary phase in a selective medium comprising: 20 g/l glucose, 6.7 g/l of the medium “yeast nitrogen base without amino acids”, 1.85 g/l of the selective medium “Yeast synthetic drop-out medium without Uracil and tryptophan”. The medium “Yeast synthetic drop-out medium without Uracil and tryptophan” is composed of:
76 mg of each of the following amino acids: Alanine, Arginine, Asparagine, Aspartic acid, Cysteine, Glutamic acid, Glutamine, Glycine, Histidine, myo-Inositol, Isoleucine, Lysine, Methionine, Phenylalanine, Proline, Serine, Threonine, Tyrosine, Valine
380 mg of Leucine
18 mg of Adenine
8 mg of para-aminobenzoic acid.

The recombinant yeasts were then centrifuged for 5 minutes at 4000 rpm, washed in PBS (Phosphate-Buffered Saline), then resuspended in 20 g/l galactose, 6.7 g/l of the medium “yeast nitrogen base without amino acids” and 1.85 g/l of the selective medium “Yeast synthetic drop-out medium without Uracil and tryptophan”. The yeasts were cultured at 30° C. for 48 h for the induction of the expression of the recombinant proteins by galactose. The recombinant yeasts were then centrifuged for 5 minutes at 4000 rpm, washed in PBS (Phosphate-Buffered Saline), and were then treated in the following manner:
a) Resuspension in a mixture of 50% ethanol, 50% water v/v for 25 minutes and further washing in PBS (permeabilization with ethanol);
b) Resuspension in a mixture of 50% isopropanol, 50% water v/v for 25 minutes and further washing in PBS (permeabilization with isopropanol);
c) Resuspension in a 2% solution of paraformaldehyde (PFA) for 10 minutes, washing in PBS, then resuspension in a mixture of 50% ethanol, 50% water v/v for 25 minutes and further washing with PBS (fixing with PFA then permeabilization with ethanol);
d) Resuspension in a 2% solution of paraformaldehyde (PFA) for 10 minutes, washing in PBS, then resuspension in a mixture of 50% isopropanol, 50% water v/v for 25 minutes and further washing with PBS (fixing with PFA then permeabilization with isopropanol); or
e) Resuspension in a mixture of 50% ethanol, 50% water v/v for 25 minutes and further washing with PBS, then resuspension in a 2% solution of paraformaldehyde (PFA) for 10 minutes and further washing with PBS (permeabilization with ethanol then fixation with PFA).

The tumor antigens that were used in the constructs are:
AH1A5 (modification of GP70 423-431): the non-mutated antigen AH-1 (SPSYVYHQF, SEQ ID NO: 20) is the immunodominant H-2Ld antigen derived from gp70 423-431, which elicits a CD8+ response against the murine colorectal cancer line CT26 (Enhanced Antigen-Specific Antitumor Immunity with Altered Peptide Ligands that Stabilize the MHC-Peptide-TCR Complex, Slansky et al, Immunity, Vol 13, 529-538, October, 2000). Gp70 is silent in most normal tissues in mice. Despite the induction of CD8+, the native AH-1 antigen does not enable immunization against the CT26 tumor because of a low affinity of this epitope for the TCR. A cryptic peptide of AH-1 (modification of GP70 423-431) is thus used which makes it possible to increase its affinity for the TCR and to immunize the mouse by the MHCI pathway. The tumor antigen that was used in this example is the cryptic peptide of sequence SPSYAYHQF (SEQ ID NO: 15) (AH1A5).
TRP2 (180-188): Tyrosinase-related protein 2 (TRP2) is a protein involved in the pigmentation of melanocytes that plays a role in the progression of melanoma. The tumor antigen that was used in this example is the cryptic peptide of sequence SVYDFFVWL (SEQ ID NO: 17) (TRP2).
TRP1 (455-463): Tyrosinase-related protein 1 (TRP1), also known as glycoprotein gp75 (Tyrp1/gp75), is a protein involved in the pigmentation of melanocytes that plays a role in the progression of melanoma. The tumor antigen that was used in this example is the cryptic peptide of sequence TAPDNLGYM (SEQ ID NO: 16) (TRP1).

A G4S linker was placed between the anchoring polypeptide and the tumor antigen. This linker is an amino acid chain composed of 4 consecutive glycines and one serine. A G4S linker was also placed between each of the antigens of the yeast expressing the OVA1-TRP1-TRP2 antigens. A protein tag, c-myc, was also added between the anchoring polypeptide and the tumor antigen to enable the detection of the complexes at the surface of the yeasts. A targeting polypeptide ScFv anti-DEC205 was also added, as well as an additional secretion signal peptide at the N-terminus of the targeting polypeptide ScFv anti-DEC205 using the addressing polypeptide encoded by the nucleic sequence “prepro alpha factor leader peptide” derived from the yeast pheromone Mf(alpha)1p (SEQ ID No. 12).

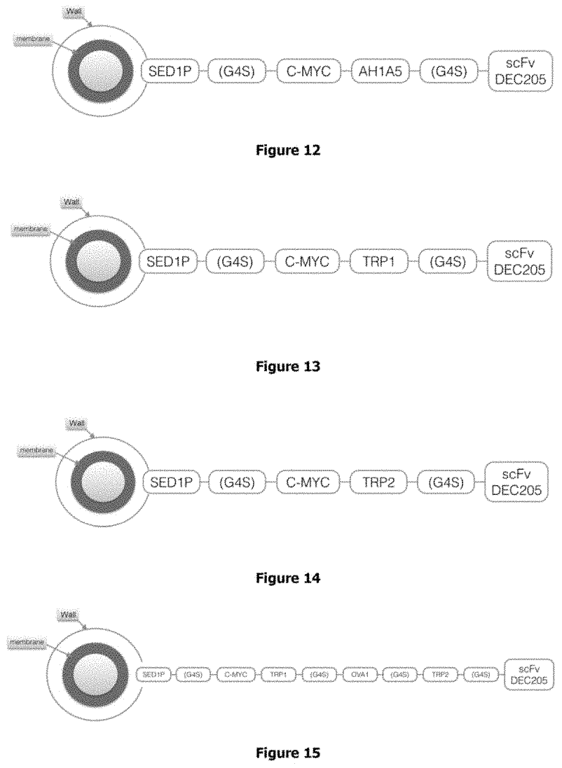

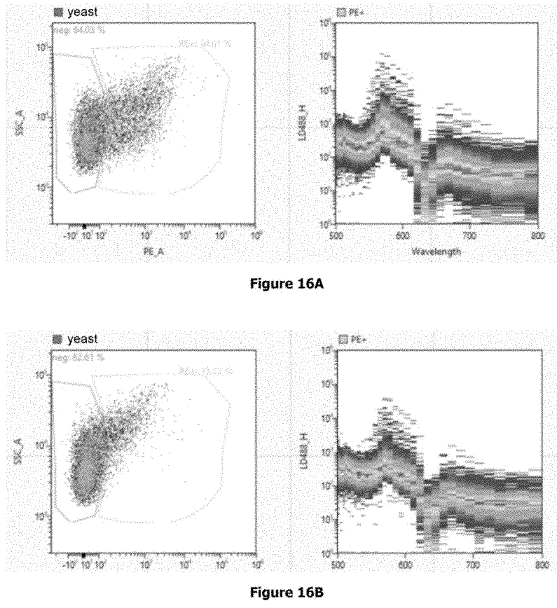

Four types of genetically modified yeasts were thus obtained:
1) A yeast which expresses, at its wall, the fusion protein [scFv DEC205]-[G4S]-[AH1A5]-[C-MYC]-[G4S]-[SED1P] (FIG. 12);
2) A yeast which expresses, at its wall, the fusion protein [scFv DEC205]-[G4S]-[TRP1]-[C-MYC]-[G4S]-[SED1P] (FIG. 13);
3) A yeast which expresses, at its wall, the fusion protein [scFv DEC205]-[G4S]-[TRP2]-[C-MYC]-[G4S]-[SED1P] (FIG. 14); and
4) A yeast which expresses, at its wall, the fusion protein [scFv DEC205]-[G4S]-[TRP2]-[G4S]-[OVA1]-[G4S]-[TRP1]-[C-MYC]-[G4S]-[SED1P] (FIG. 15).

### Example 8: Monitoring the Expression of the Fusion Proteins at the Surface of the Yeasts

The four types of genetically modified yeasts obtained in example 7, expressing the tumor antigens AH1A5, TRP1, TRP2 or OVA1-TRP1-TRP2, were assayed with cytometry to confirm that they were indeed expressing the tumor antigens at their wall.

After the induction of the yeasts in galactose, the yeasts at a concentration of 1×10⁷ cells per ml were suspended in a 2% PFA solution for 10 minutes. The yeasts were then washed in PBS containing 1% BSA. After washing, they were cultured for 1 hour at room temperature with an anti-c-myc primary antibody at a ratio of 1:100 (murine primary IgG2a antibody, MA1-16637, ThermoFisher). After 3 washes in PBS containing 1% BSA, the yeasts were incubated for 1 hour at room temperature with an anti-IgG2a secondary antibody at a ratio of 1:50 (murine secondary IgG2a antibody, 31863, ThermoFisher) and underwent spectral flow cytometry in the PE preferential channel.

The anti-c-myc antibody makes it possible to detect, by spectral flow cytometry, the yeasts which are expressing each of the fusion proteins at the wall. Under the experimental conditions tested, the labeling antibody of the fusion protein was not able to penetrate inside the yeasts, which made it possible to highlight the antigens oriented toward the external environment of the yeast and not toward the periplasm of the yeast.

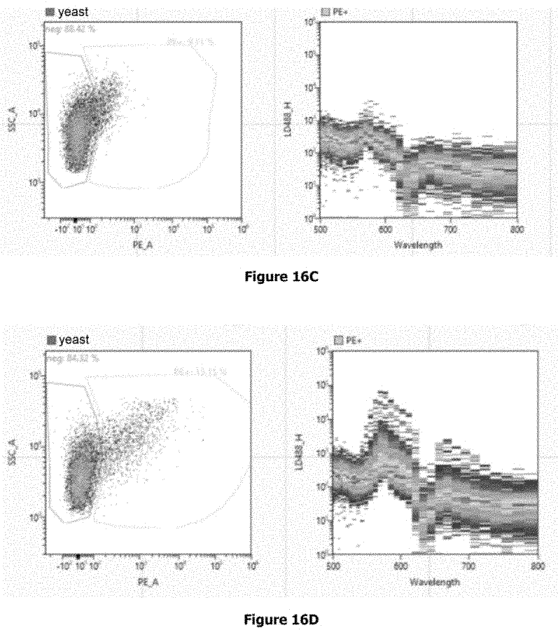

The results for the four types of genetically modified yeasts are presented in FIG. 16:
FIG. 16A: 34% of the yeasts express the fusion protein [scFv DEC205]-[G4S]-[AH1A5]-[C-MYC]-[G4S]-[SED1P] at their wall;
FIG. 16B: 15% of the yeasts express the fusion protein [scFv DEC2O5]-[G4S]-[TRP2]-[G4S]-[OVA1]-[G4S]-[TRP1]-[C-MYC]-[G4S]-[SED1P] at their wall;
FIG. 16C: 9% of the yeasts express the fusion protein [scFv DEC205]-[G4S]-[TRP2]-[C-MYC]-[G4S]-[SED1P] at their wall;
FIG. 16D: 13% of the yeasts express the fusion protein [scFv DEC205]-[G4S]-[TRP1]-[C-MYC]-[G4S]-[SED1P] at their wall.

The figures show the expression of the tumor antigens AH1A5, TRP1, TRP2 and OVA1-TRP1-TRP2 at the wall of the yeasts obtained in example 7.

Conclusion:

The spectral flow cytometry demonstrated on the one hand the effective anchoring of the fusion proteins comprising a tumor antigen at the wall of the yeasts and on the other hand the effective anchoring of a fusion protein comprising several tumor antigens at the wall of the yeasts.

### Example 9: Assay for Characterization of a Permeabilized Yeast

The assay for characterization of a permeabilized yeast uses an enzyme naturally present in the cytosol of the yeast, alkaline phosphatase. Its substrate, para-nitrophenyl phosphate (pNPP) assumes a yellow color which can be read by absorbance at 405 nm after hydrolysis, proportionally to the enzymatic activity (A rapid method for determination of acid phosphatase activity of whole yeast cells, Galabova et al, June 2008, Letters in Applied Microbiology 16 (3):161-163). When the yeast has been permeabilized, the pNPP substrate enters the yeast and is hydrolyzed by the alkaline phosphatase, which generates an absorbance signal at 405 nm. Yeast that has not been permeabilized is not permeable to pNPP.

The wild-type S. cerevisiae yeasts were cultured in yeast complete medium until saturation at 30° C. with stirring at 250 rpm in an Erlenmeyer flask. The yeasts were harvested by centrifugation at 3000 g for 5 minutes then washed once with PBS, and resuspended in PBS at optical density OD=1 at 600 nm. Different samples of yeasts were then subjected to the following treatments:
living: no treatment;
PFA: fixation in a 2% PFA solution for 10 minutes (PFA);
PFA+ethanol: fixation in a 2% PFA solution for 10 minutes, washing with PBS then permeabilization in a 50% ethanol solution v/v for 25 minutes;
Ethanol+PFA: permeabilization in a 50% ethanol solution v/v for 25 minutes, washing with PBS then fixation in a 2% PFA solution for 10 minutes;
PFA+isopropanol: fixation in a 2% PFA solution for 10 minutes, washing with PBS then permeabilization in a 50% isopropanol solution v/v for 25 minutes;
Isopropanol: permeabilization in a 50% isopropanol solution v/v for 25 minutes;
Ethanol: permeabilized in a 50% ethanol solution v/v for 25 minutes; or
UV: irradiation twice at 999 J/cm² with homogenization between the two irradiations in an HL-2000 HybriLinker, making it possible to carry out UV cross-linking.

100 μl of solution of the different samples of yeasts treated as described above were taken and mixed with 150 μl of para-nitrophenyl phosphate solution (P7998 SIGMA-Alkaline Phosphatase Yellow (pNPP) Liquid Substrate System for ELISA). The mixture was incubated for 30 minutes at room temperature. The reaction was stopped after 30 minutes with 35 μl of 3 M NaOH. The yeasts were centrifuged for 5 minutes at 4000 rpm, then 100 μl of supernatant was taken off to read its absorbance at 405 nm. The blank used was pNPP alone.

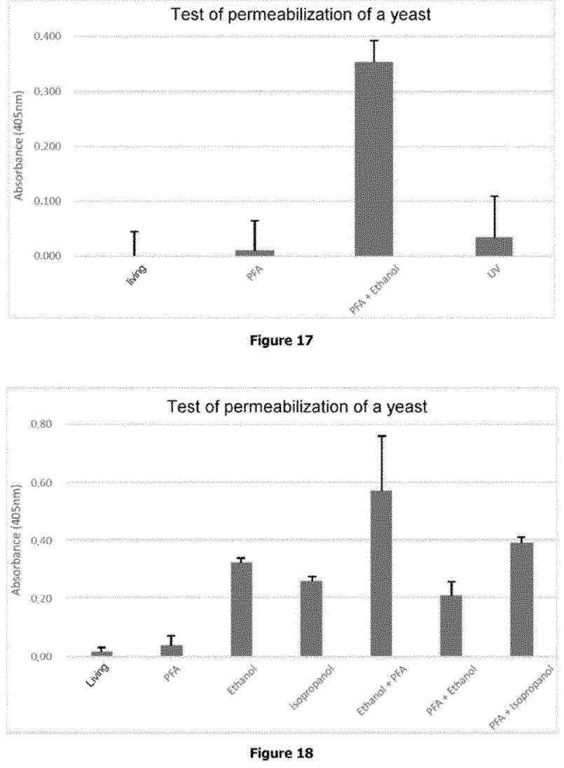

The results are presented in FIGS. 17 and 18.

Conclusion: The assay confirmed that treatment with isopropanol or ethanol made it possible to permeabilize the yeasts, with and without fixation with PFA. The assay also demonstrated that irradiation with UV or fixation with PFA did not make it possible to permeabilize the yeasts.

### Example 10: Measurement of the Activation of the Cytotoxic CD8+ T Lymphocytes With a Yeast Having the OVA1 Antigen at its Wall and Having Undergone Different Permeabilization Treatments

The activation of the cytotoxic CD8+ T lymphocytes was measured using a colorimetric test using beta-galactosidase and one of its substrates, CPRG (Chlorophenol red-beta-galactopyranoside), as described in example 4.

Materials: dendritic cells, CD8+ T lymphocytes and a non-permeabilized genetically modified yeast which expresses an antigen at its wall.

The dendritic cells used were murine dendritic cells derived from the MutuDC line (obtained from the University of Lausanne). This line originates from immortalized splenic CD8α dendritic cells that retain the ability to cross-present an antigen and to activate killer lymphocytes (KL). The cells of the MutuDC line were cultured in RPMI-1640 medium supplemented with 10% FCS, HEPES, 50 μM 2-mercaptoethanol, 50 U/ml penicillin, and 50 μg/ml streptomycin, at 37° C. with 5% CO₂.

The CD8+ T lymphocytes used originated from the B3Z hybridoma. The B3Z hybridoma, a line specific for the peptide OVA 257-264 (SIINFEKL, SEQ ID NO: 21), was provided by the Institut Curie (Paris V). These cells have the particular feature of producing beta galactosidase under the control of an IL-2 (interleukin 2) promoter. The B3Z cells were cultured in RPMI-1640 medium supplemented with 10% FCS, Glutamax, HEPES, 50 μM 2-mercaptoethanol, 50 U/ml penicillin, and 50 μg/ml streptomycin, at 37° C. with 5% CO₂. The dendritic cells were distributed at an amount of 100 000 cells per well in 96-round-bottom-well plates.

The non-permeabilized genetically modified yeast expressed the OVA1 antigen at its wall, fused to scFv DEC205 (scFv DEC205-OVA1-SED) and was prepared according to example 2 (FIG. 4). This yeast underwent the following treatments:
living: no treatment;
PFA: fixation in a 2% PFA solution for 10 minutes (PFA);
PFA+ethanol: fixation in a 2% PFA solution for 10 minutes, washing with PBS then permeabilization in a 50% ethanol solution v/v for 25 minutes;
Ethanol+PFA: permeabilization in a 50% ethanol solution v/v for 25 minutes, washing with PBS then fixation in a 2% PFA solution for 10 minutes;
PFA+isopropanol: fixation in a 2% PFA solution for 10 minutes, washing with PBS then permeabilization in a 50% isopropanol solution v/v for 25 minutes;
Isopropanol: permeabilization in a 50% isopropanol solution v/v for 25 minutes;
Ethanol: permeabilized in a 50% ethanol solution v/v for 25 minutes; or
UV: irradiation twice at 999 J/cm² with homogenization between the two irradiations in an HL-2000 HybriLinker, making it possible to carry out UV cross-linking.

then cultured with the cells of the MutuDC line for 5 h at a ratio of 30:1 (MOI 30, for Multiplicity of infection),

The lymphocytes of the B3Z line were subsequently added at an amount of 100 000 per well for 18 h at 37° C. and 5% CO₂. The plates were then washed and the activity of the beta galactosidase produced by the CD8+ T lymphocytes was measured after addition of 120 μl of lysis buffer (which contains PBS, 9 mM MgCl₂, 0.125% NP40 and 0.15 mM chlorophenol red-beta-galactopyranoside). After the color change to red, the absorbance in the red at 575 nm was read using a ClarioStar plate reader.

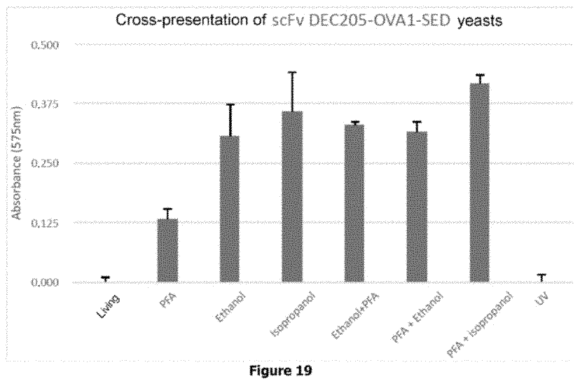

The results are presented in FIG. 19.

Conclusion: This cross-presentation test showed that the permeabilized yeasts (inactivated or not) were able to significantly activate the killer lymphocytes. The activation obtained with the permeabilized yeasts was much greater than the activation obtained with the non-permeabilized yeasts (PFA or UV). It should especially be noted that yeasts treated with UV were not able to activate the killer lymphocytes, like those yeasts that did not undergo any treatment (living).

The content of the ASCII text file of the sequence listing named Substitute-Sequence-Listing-21721-0701, having a size of 16.8 kb and a creation date of 10 Mar. 2020, and electronically submitted via EFS-Web on 10 Mar. 2020, is incorporated herein by reference in its entirety.

1. A process for preparing an immunotherapeutic yeast, said process comprising the steps of:
a) obtaining a genetically modified yeast which expresses, at its wall, one or more tumor antigen(s) and optionally a polypeptide or a protein for targeting dendritic cells;
b) permeabilizing the genetically modified yeast in order to obtain an immunotherapeutic yeast, optionally inactivating the yeast before or after the permeabilization step.

2. The process of claim 1, wherein step a) is: obtaining a genetically modified yeast which expresses, at its wall, one or more fusion proteins of formula (Ia):
[protein or polypeptide for targeting dendritic cells]n-[tumor antigen]x-[peptide linker]m-[polypeptide or protein for anchoring to the wall of the genetically modified yeast] (Ia);
n is equal to 0 or 1, m is equal to 0 or 1, and x is an integer ranging from 1 to 300, preferably ranging from 1 to 50.

3. The process of claim 1, wherein step a) comprises the steps of:
a1) introducing, into a yeast, one or more vector(s) each comprising a nucleic sequence of formula (IIa) or (IIb):
[nucleic sequence encoding a polypeptide for addressing the wall of the genetically modified yeast]-[nucleic sequence encoding a polypeptide or a protein for targeting dendritic cells]n-[nucleic sequence encoding a tumor antigen]x-[nucleic sequence encoding a peptide linker]m-[nucleic sequence encoding a polypeptide or a protein for anchoring to the wall of the genetically modified yeast] (IIa),
n is equal to 0 or 1, m is equal to 0 or 1, and x is an integer ranging from 1 to 300, preferably ranging from 1 to 50;
[nucleic sequence encoding a polypeptide or a protein for targeting dendritic cells]n-[nucleic sequence encoding a tumor antigen]x-[nucleic sequence encoding a peptide linker]m-[nucleic sequence encoding a polypeptide or a protein for anchoring to the wall of the genetically modified yeast]-[nucleic sequence encoding a polypeptide for addressing the wall of the genetically modified yeast] (IIb),
n is equal to 0 or 1, m is equal to 0 or 1, and x is an integer ranging from 1 to 300, preferably ranging from 1 to 50;
in order to obtain a genetically modified yeast capable of expressing, at its wall, one or more fusion protein(s) of formula (Ia):
[protein or polypeptide for targeting dendritic cell s]n-[tumor antigen]x-[peptide linker]m-[polypeptide or protein for anchoring to the wall of the genetically modified yeast] (Ia),
n is equal to 0 or 1, m is equal to 0 or 1, and x is an integer ranging from 1 to 300, preferably ranging from 1 to 50; and
a2) culturing the genetically modified yeast under conditions suitable for the expression of the fusion protein(s) at the wall of the genetically modified yeast.

4. The preparation process of claim 1, wherein the yeast is chosen from the genus Saccharomyces, Schizosaccharomyces, Kluveromyces, Ogataea. or Candida, preferably the genus Saccharomyces; preferably, the yeast is Saccharomyces cerevisiae.

5. The preparation process of claim 1, wherein the anchoring polypeptide or protein is a yeast polypeptide or protein expressed at the wall of the yeasts, preferably chosen from Aga2p or Sed1p.

6. The preparation process of claim 1, wherein the genetically modified yeast is permeabilized with a water/ethanol mixture.

7. The preparation process of claim 1, wherein the polypeptide or protein for targeting dendritic cells is chosen from an antibody able to specifically bind to the protein DEC-205, a fragment of antibody able to specifically bind to the protein DEC-205, the plasminogen activator (PLA) of the bacterium Yersinia pestis or a sequence derived from the plasminogen activator (PLA) of the bacterium Yersinia pestis.

8. The process of claim 1, wherein the tumor antigen(s) is (are) chosen from Melan-A, Tyrosinase, gp100, MAGEA1, MAGEA10, MAGEA11, MAGEA12, MAGEA2, MAGEA2B, MAGEA3, MAGEA4, MAGEA6, MAGEA8, MAGEA9, MAGEB1; MAGEB10, MAGEB16, MAGEB18, MAGEB2, MAGEB3, MAGEB4, MAGEB5, MAGEB6, MAGEB6B, New York Esophageal 1 antigen (NY-ESO-1), MAGEC1, MAGEC2, L-antigen (LAGE), TRP-1, TRP-2, P53, KRAS, CEA, WT1, MUC1, SART3, SURVIVIN 2B, RNF43/TOMM34, TGFBRII, HER2/neu, BRAF, PI3K, APC, BAX, beta-2 microglobulin, telomerase or NRAS.

9. An immunotherapeutic yeast able to be obtained by carrying out the process of claim 1.

10. A genetically modified and permeabilized immunotherapeutic yeast which expresses, at its wall:
(i) one or more tumor antigen(s), preferably chosen from Melan-A, Tyrosinase, gp100, MAGEA1, MAGEA10, MAGEA11, MAGEA12, MAGEA2, MAGEA2B, MAGEA3, MAGEA4, MAGEA6, MAGEA8, MAGEA9, MAGEB1; MAGEB10, MAGEB16, MAGEB18, MAGEB2, MAGEB3, MAGEB4, MAGEB5, MAGEB6, MAGEB6B, New York Esophageal 1 antigen (NY-ESO-1), MAGEC1, MAGEC2, L-antigen (LAGE), TRP-1, TRP-2, P53, KRAS, CEA, WT1, MUC1, SART3, SURVIVIN 2B, RNF43/TOMM34, TGFBRII, HER2/neu, BRAF, PI3K, APC, BAX, beta-2 microglobulin, telomerase or NRAS; and
(ii) optionally a polypeptide or protein for targeting dendritic cells.

11. The yeast of claim 10, which expresses, at its wall, a fusion protein of formula (Ia):
[protein or polypeptide for targeting dendritic cells]n-[tumor antigen]x-[peptide linker]m-[polypeptide or protein for anchoring to the wall of the genetically modified yeast] (Ia);
n is equal to 0 or 1, m is equal to 0 or 1, and x is an integer ranging from 1 to 300, preferably ranging from 1 to 50.

12. The yeast of claim 10, wherein the polypeptide or protein for targeting dendritic cells is chosen from an antibody able to specifically bind to the protein DEC-205, a fragment of antibody able to specifically bind to the protein DEC-205, the plasminogen activator (PLA) of the bacterium Yersinia pestis or a sequence derived from the plasminogen activator (PLA) of the bacterium Yersinia pestis.

13. An immunotherapeutic composition comprising a yeast obtainable by the process of claim 1, and a pharmaceutically acceptable carrier.

14.-17. (canceled)

18. An immunotherapeutic composition comprising a yeast obtainable by carrying out the process of claim 1, a pharmaceutically acceptable carrier, and a therapeutic agent.

19. An immunotherapeutic composition comprising a yeast of claim 10 and a pharmaceutically acceptable carrier.

20. An immunotherapeutic composition comprising a yeast of claim 10, a pharmaceutically acceptable carrier, and a therapeutic agent.

21. A medicament comprising an immunotherapeutic yeast obtainable by carrying out the process of claim 1.

22. A medicament comprising an immunotherapeutic yeast of claim 10.

23. A medicament comprising an immunotherapeutic composition, said immunotherapeutic composition comprising a yeast obtainable by carrying out the process of claim 1, and a pharmaceutically acceptable carrier.

24. A medicament comprising an immunotherapeutic composition, said immunotherapeutic composition comprising a yeast obtainable by carrying out the process of claim 1, a pharmaceutically acceptable carrier, and a therapeutic agent.

25. A medicament comprising an immunotherapeutic composition, said immunotherapeutic composition comprising the yeast of claim 10 and a pharmaceutically acceptable carrier.

26. A medicament comprising an immunotherapeutic composition, said immunotherapeutic composition comprising a yeast as claimed in claim 10, a pharmaceutically acceptable carrier, and a therapeutic agent.

27. Method for treating or preventing cancer comprising administering an immunotherapeutic yeast obtainable by carrying out the process of claim 1.

28. Method for treating or preventing cancer comprising administering the yeast of claim 10.

29. Method for treating or preventing cancer comprising administering an immunotherapeutic composition, said immunotherapeutic composition comprising a yeast obtainable by carrying out the process of claim 1, and a pharmaceutically acceptable carrier.

30. Method for treating or preventing cancer comprising administering an immunotherapeutic composition, said immunotherapeutic composition comprising a yeast obtainable by carrying out the process of claim 1, a pharmaceutically acceptable carrier, and a therapeutic agent.

31. Method for treating or preventing cancer comprising administering an immunotherapeutic composition, said immunotherapeutic composition comprising the yeast of claim 10 and a pharmaceutically acceptable carrier.

32. Method for treating or preventing cancer comprising administering an immunotherapeutic composition, said immunotherapeutic composition comprising the yeast of claim 10, a pharmaceutically acceptable carrier, and a therapeutic agent.

33. Method for treating or preventing solid cancer comprising administering an immunotherapeutic yeast obtainable by carrying out the process of claim 1.

34. Method for treating or preventing solid cancer comprising administering the yeast of claim 10.

35. Method for treating or preventing solid cancer comprising administering an immunotherapeutic composition, said immunotherapeutic composition comprising a yeast obtainable by carrying out the process of claim 1, and a pharmaceutically acceptable carrier.

36. Method for treating or preventing solid cancer comprising administering an immunotherapeutic composition, said immunotherapeutic composition comprising a yeast obtainable by carrying out the process of claim 1, a pharmaceutically acceptable carrier, and a therapeutic agent.

37. Method for treating or preventing solid cancer comprising administering an immunotherapeutic composition, said immunotherapeutic composition comprising the yeast of claim 10 and a pharmaceutically acceptable carrier.

38. Method for treating or preventing solid cancer comprising administering an immunotherapeutic composition, said immunotherapeutic composition comprising the yeast of claim 10, a pharmaceutically acceptable carrier, and a therapeutic agent.

39. Method for treating or preventing melanoma or colon cancer comprising administering an immunotherapeutic yeast obtainable by carrying out the process of claim 1.

40. Method for treating or preventing melanoma or colon cancer comprising administering the yeast of claim 10.

41. Method for treating or preventing melanoma or colon cancer comprising administering an immunotherapeutic composition, said immunotherapeutic composition comprising a yeast obtainable by carrying out the process of claim 1, and a pharmaceutically acceptable carrier.

42. Method for treating or preventing melanoma or colon cancer comprising administering an immunotherapeutic composition, said immunotherapeutic composition comprising a yeast obtainable by carrying out the process of claim 1, a pharmaceutically acceptable carrier, and a therapeutic agent.

43. Method for treating or preventing melanoma or colon cancer comprising administering an immunotherapeutic composition, said immunotherapeutic composition comprising the yeast of claim 10 and a pharmaceutically acceptable carrier.

44. Method for treating or preventing melanoma or colon cancer comprising administering an immunotherapeutic composition, said immunotherapeutic composition comprising the yeast of claim 10, a pharmaceutically acceptable carrier, and a therapeutic agent.


SEQUENCE LISTING (21 sequences)

SEQ ID NO: 1 - DNA, 464 bp, Artificial Sequence
  GAL1 promoter
aggtagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactataatg

SEQ ID NO: 2 - DNA, 318 bp, Artificial Sequence
  CYC1 terminator
aaacgacttaattacctacttgaaaaaagcctgtgagtaaacaggccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaacgttttgggacgctcgaaggctttaatttgcaagct

SEQ ID NO: 3 - DNA, 66 bp, Artificial Sequence
  T27 terminator
tgggtggtatatatatatatatatatatatataactgtctagaaataaagagtatcatctttcaaa

SEQ ID NO: 4 - DNA, 1019 bp, Artificial Sequence
  URA3
tggcttttcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtttctacaggatctgacattattattgttggaagaggactatttgcaaagggaagggatgctaaggtagagggtgaacgttacagaaaagcaggctgggaagcatatttgagaagatgcggccagcaaaac

SEQ ID NO: 5 - DNA, 2217 bp, Artificial Sequence
  pSB1K3
actagtggtctccagcgtcttcctgcagtccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgagtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagctcgagtgccacctgacgtctaagaaaccattattatcatgacattaacctataaaaataggcgtatcacgaggcagaatttcagataaaaaaaatccttagctttcgctaaggatgatttctggaattcgaagacgcttgagacctctaga

SEQ ID NO: 6 - DNA, 569 bp, Artificial Sequence
  XI-2 UP
gaggattttcgatggagcaggatgaggagaaatagtaccacatgtatatatccattacaaaaaggtttatatacaattacaatagacccttgttggggtttctgaaaaaagaagtagtcgatgccatcggcaataatacggaattacgagaaacacaatcccgatccttttttgggtaattacttcaccgattctaccgatttatcatgccaaaaaaaattcaccgtgggttctagaagtgccctttgaggattgtagccactctaacccacacggcctccttactagctgactaaggtgacaaaaccgcaaggactggaaagtcgccactcatctgaaaattctcaagtttttcactactgagtttatgctttcgaatttttttgttcggtaatagcacggcggttcgattcaattccgccgctccgagcgatgctccgcaaaactcagtaataagctttctgatggttcaccccttttttagcacgcggggtgtaactcaacagaaaaatgtgccatagaacaagactaggcaaaagccaaggagcgtttgccatgaacttccacaa

SEQ ID NO: 7 - DNA, 563 bp, Artificial Sequence
  XI-2 DOWN
cacacgactagcgctttcagatattaaaaagtttagatgtaggttttagcggtaacagttatataaatcgtgtttcttctcttgatgaaacaaaaaaatgctagaaaaactttgtcgtttcttacttttggtgcgctttgcagttttcgtggctagacttagaatcatttctcctcagatttcttgattaaagtttggtgcgaagccctactctaacattggtgttcttcttttcattcacgcaagttaagtccaggaaggtgagcaaatgctcatccttctgttcatgcgtgacggctgaattatccttatctggcgtacccgtgcagccgtttccgtgcctcggttcctccgagatatccttagggaccgccagggaccatgattgcgtcaactgttgtcaccgctccagaggatcctctgtaaccttttcaaccataaaaatagaaagggcacagggttggggtatttgaattttttttttgggttttttcgtattacttattacttaccttcttttctatataattttgtttttccctggtagagcggaatcttcccac

SEQ ID NO: 8 - DNA, 675 bp, Artificial Sequence
  TRP1
atgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaatag

SEQ ID NO: 9 - DNA, 2178 bp, Artificial Sequence
  AGA1P
atgacattatctttcgctcattttacctacctgttcacaatattgttgggattaactaatattgccttggcatctgatccagaaacgattctagtgacgataaccaagacaaacgatgcaaatggggttgttacaactacagtttcacccgcgctagtctccacatccactatcgttcaagctggcactacgacattgtatacgacttggtgtccattgacggtatccacttcatctgctgccgaaataagtccttcaatatcgtacgctactaccctatccagatttagtactttgacattatctacagaagtctgctcccatgaggcatgtccttcgtcatcgacgttgccaaccaccaccttatctgtgacttccaagttcacttcatatatttgccctacttgtcacacaaccgctatcagctcattatccgaagtaggaactacaaccgtggtatcatccagcgccattgaaccatcaagtgcctctataatctcacctgtcacctctacactttcgagtacaacatcgtccaatccaactactacctccctaagttcgacatctacatctccaagctctacatctacatctccaagctctacatctacctcatcaagttcgacatctacctcatcaagttcgacatctacctcatcaagttcgacatctacatctccaagttcgacatccacatcttcaagtttgacatccacatcttcaagttctacatctacatcccaaagttctacatctacctcatcaagttcgacatctacatctccaagctctacatctacctcatcaagttcaacatctacatctccaagttctaaatctacttctgcaagctccacttccacttcttcatattcaacatctacatccccaagtttgacttcttcatctccaactttggcttccacttctccaagttcaacatctattagctctacttttactgattcaacttcatcccttggctcctctatagcatcttcatcaacgtctgtgtcattatacagcccatccacacctgtttactccgtcccttcgacttcgtcaaatgttgcaactccttctatgacttcttcaactgttgaaacaactgttagttcacaaagttcgtctgaatatatcaccaaatcctcaatttctactactatcccatcattttccatgtctacatatttcaccactgttagtggagtcactacaatgtatacgacatggtgtccttatagctctgaatctgagactagcacattaaccagtatgcatgaaacggttacaacagacgctacagtctgcactcacgagtcttgcatgccctcgcagacaacaagtttgattacatcttctataaaaatgtccactaaaaacgtcgcaacttctgtaagcacctcaacggttgaatcctcatatgcatgctccacatgtgctgaaacgtcacactcgtattcttccgtgcaaacagcttcatcaagttctgtaacacagcagaccacatccacaaagagttgggtaagttcaatgacaacttcggatgaagatttcaataagcacgctaccggtaagtatcatgtaacatcttcaggtacctcaaccatttcgactagtgtaagtgaagccacgagtacatcaagcattgactcagaatctcaagaacaatcatcacacttattatcgacatcggtcctttcatcctcctccttgtctgctacattatcctctgacagtactattttgctattcagttctgtatcatcactaagtgtcgaacagtcaccagttaccacacttcaaatttcttcaacatcagagattttacaacccacttcttccacagctattgctacaatatctgcctctacatcatcactttccgcaacatctatctctacaccatctacctctgtggaatcgactattgaatcttcatcattgactccgacggtatcttctattttcctctcatcatcatctgctccctcttctctacaaacatctgttaccactacagaagtttccactacttcaatctccatacaataccaaacttcatcaatggtaacaattagccaatatatgggcagtggatcgcaaacgcgtttgccattaggaaagttggtcttcgccatcatggcagttgcttgcaatgtaattttcagttaa

SEQ ID NO: 10 - DNA, 258 bp, Artificial Sequence
  AGA2P
caactacttaggtgtttttctattttttcagtgatcgcctcagtcttagcccaggagcttacgactatctgtgagcaaattccgagtcctacgttggagtctactccctactccttatctacgactacgattctggcgaatgggaaggccatgcagggtgtctttgaatactataagtcagtgacttttgtgtccaactgcggctcgcacccctctactacgtcaaagggtagtcctattaatactcaatatgtcttt

SEQ ID NO: 11 - DNA, 960 bp, Artificial Sequence
  SED1P
ttttccaatagtacttctgcttcttccaccgacgtcacttcctcctcttccatctccacttcctctggctcagtaactatcacttcttctgaagctccagaatccgataacggtaccagcactgctgcaccaactgaaacctcaactgaggctccaaccactgctatcccaactaacggtacctctactgaagctccaaccactgctatcccaactaacggtacctctactgaagctccaactgatactactactgaagctccaaccaccgctcttccaactaacggtacttctactgaagctccaactgatactactactgaagctccaaccaccggtcttccaaccaacggtaccacttcagctttcccaccaactacttctttgccaccaagcaatactaccaccactcctccttataacccatctactgactataccactgactatactgtagtcactgaatatactacttactgtccagaaccaaccactttcaccactaacggtaagacttataccgtcactgaaccaaccactttgactatcactgactgtccatgcaccattgaaaagccaactaccacttcaaccaccgaatatactgtagtcactgagtatactacttactgtccagaaccaaccactttcaccactaacggtaagacttataccgtcactgaaccaaccactttgactatcactgactgtccatgtactattgaaaagagcgaagcccctgagtcttctgtcccagttaccgaatctaagggcactaccaccaaagaaactggtgttactaccaagcaaaccactgccaacccaagtctaaccgtctccactgtcgtcccagtttcatcctctgcttcttctcattccgttgtcatcaatagtaacggtgctaacgtcgtcgttccaggtgctttaggtttggctggtgttgctatgttattcttataa

SEQ ID NO: 12 - DNA, 264 bp, Artificial Sequence
  prepro Mf(alpha)
aggttcccgagcattttcacggccgtgctatttgctgcgtctagtgcattggcggctcccgtcaacactaccactgaggatgaaactgcacagattcctgctgaggctgtcatcggttatcttgatttggagggcgattttgatgtcgcagtgttgccattctccaacagtacaaacaacggacttttattcatcaacaccacgatagcctctatagcggcgaaggaagaaggtgtgtctttagacaagcgtgaagcagaggca

SEQ ID NO: 13 - DNA, 846 bp, Artificial Sequence
  anti-DEC205 scFv
ggtgataatgatatccactttgcctttctctccactggtgtgcactcggatattgtgctgacccagtctccgtcatttctgtctacttctcttggaaatagcatcaccatcacttgccatgccagtcagaatatcaagggttggttagcctggtaccagcaaaagtcagggaatgctcctcaactgttgatttataaggcatctagcctgcaatcaggggttccatcaagattcagtggcagtggatctggaactgattatattttcactatcagcaaccttcagcctgaagatattgccacttattactgtcagcattatcaaagctttccgtggacgttcggtggaggcaccaagctggaattgaaagggtcgaccagcggttctgggaaaccaggttccggtgaaggctcgagcaaaggtgaggtgaaactgttggaatctggaggaggtttggttcagccggggggttctctgagactctcctgtgcagcttctggattcaccttcaatgatttctatatgaactggatccgccagcctccagggcaggcacctgagtggttgggtgttattagaaataaaggtaatggttatactactgaggtcaatacttctgtgaaggggcggttcaccatctccagagataatacccaaaatatcctctatcttcaaatgaatagcctgagagctgaggataccgccatttactactgtgcaagaggcggtccttattactatagtggtgacgacgccccttactggggccaaggagtcatggtcactgtctcctccggagctagcggcaaaccaatcccaaacccactgctgggcctggataccggt

SEQ ID NO: 14 - protein, 19 aa, Artificial Sequence
  MEQLESIINFEKLTEWTSA
MEQLESIINFEKLTEWTSA

SEQ ID NO: 15 - protein, 9 aa, Artificial Sequence
  AH1A5 antigen
SPSYAYHQF

SEQ ID NO: 16 - protein, 9 aa, Artificial Sequence
  TRP1 antigen
TAPDNLGYM

SEQ ID NO: 17 - protein, 9 aa, Artificial Sequence
  TRP2 antigen
SVYDFFVWL

SEQ ID NO: 18 - DNA, 11 bp, Artificial Sequence
  5′ PCR primer
ggtctctaatg

SEQ ID NO: 19 - DNA, 11 bp, Artificial Sequence
  3′ PCR primer
gagttgagacc

SEQ ID NO: 20 - protein, 9 aa, Artificial Sequence
  AH-1 antigen
SPSYVYHQF

SEQ ID NO: 21 - protein, 8 aa, Artificial Sequence
  OVA peptide 257-264
SIINFEKL